(19) United States
(12) Patent Application Publication
(10) Pub. No.: US 2013/0149695 A1
(43) Pub. Date: Jun. 13, 2013

(54) METHOD FOR DETECTING GENETIC MUTATION BY USING A BLOCKING PRIMER

(71) Applicants: Seung-Tae Lee, Seoul (KR); Chang-Seok Ki, Seoul (KR); Jong-Won Kim, Seoul (KR)

(72) Inventors: Seung-Tae Lee, Seoul (KR); Chang-Seok Ki, Seoul (KR); Jong-Won Kim, Seoul (KR)

(73) Assignee: SAMSUNG LIFE PUBLIC WELFARE FOUNDATION, Seoul (KR)

(21) Appl. No.: 13/643,990

(22) PCT Filed: Dec. 30, 2010
(86) PCT No.: PCT/KR2010/009601

§ 371 Date: Feb. 19, 2013

(30) Foreign Application Priority Data
Apr. 27, 2010 (KR) 10-2010-0039148
Nov. 29, 2010 (KR) 10-2010-0119966

(51) Int. Cl. C12Q 1/68

(52) U.S. Cl. CPC C12Q 1/6858
USPC 435/5; 435/6.11

(57) ABSTRACT

The present invention provides a method for detecting a gene mutation, comprising the step of performing PCR using generic PCR primers together with a blocking primer which competes with the generic primers and was modified at one end, and a method of diagnosing gene mutation-related diseases using the same. According to the invention, detection sensitivity and specificity can be increased by blocking the amplification of normal DNA and selectively amplifying mutant DNA.

### BACKGROUND OF THE INVENTION

1. Field of the Invention

The present invention relates to a composition for detecting mutant genes comprising generic PCR primers and a blocking primer that competes with the generic PCR primers and is modified at one end. It also discloses a method for detecting mutant genes by MEMO-PCR (mutant enrichment with terminal-modified oligonucleotide-PCR) using the composition, and also a method for diagnosing mutation-related diseases using the same.

2. Description of the Prior Art

Up to now, specific gene mutations in various tumors have been identified. Typical examples of such mutations include TP53 gene or KRAS gene mutations in various solid tumors, BRAF gene mutations in thyroid cancer and colorectal cancer, EGFR gene mutations in lung cancer and colorectal cancer, JAK2 gene mutations in chronic myeloproliferative diseases, NPM1 gene mutations in acute myelocytic leukemia, and the like. Among such mutations, a significant number of mutations tend to occur at specific locations of the gene. These mutations include TP53 Arg175His/Arg248Gln/Arg273His mutations, KRAS codon 12 and 13 mutations, BRAF Val600Glu mutation, EGFR Leu858Arg/Thr790Met mutations, JAK2 Val617Phe mutation, NPM1 exon 12 mutations, and the like.

Detection of such tumor-specific mutations is significantly useful for diagnosing cancer, deciding on a type of cancer treatment, and assessing the presence of residual tumor after treatment. Consequently, methods for detecting tumor-specific mutations have been developed and used. Typical examples thereof include direct sequencing, allele-specific PCR, restriction fragment length polymorphism (RFLP), Taqman probe, ARMS (amplification refractory mutation system)-PCR, denaturing HPLC (dHPLC), and real-time PCR assays. Assays for detecting tumor-specific mutations should have (1) a high sensitivity in detection of mutant DNA, which is present at a low concentration relative to normal DNA, and (2) a high specificity towards a mutant gene in order to minimize false-positive results caused by detecting normal DNA as mutant DNA.

However, conventional methods of detection of tumor-specific mutations did not show appropriate results in terms of sensitivity and specificity. The direct sequencing assay has the highest specificity yielding a low rate of false-positive results. However it has a shortcoming in that mutant DNA can only be detected when more than 20-30% of them are present. On the other hand, the allele-specific PCR, restriction fragment length polymorphism (RFLP) and Taqman probe assays have high sensitivity but low specificity yielding a high rate of false-positive results.

Thus, there is a high demand for the assay that has both of a high sensitivity and a high specificity towards the target gene.

Recently, a number of researches have focused on developing the modified PCR methods that allow selective amplification of mutant genes. These methods can improve greatly the sensitivity and reliability of downstream assays such as sequencing. Examples of such modified PCR methods include REMS-PCR (thermostable restriction endonuclease-mediated selective PCR) (Ward, R., et al., 1998. Restriction endonuclease-mediated selective polymerase chain reaction: a novel assay for the detection of K-ras mutations in clinical samples. Am J Pathol 153:373-379), PNA (peptide nucleic acid) (Sun, X., et al., 2002. Detection of tumor mutations in the presence of excess amounts of normal DNA. Nat Biotechnol 20:186-189) or LNA (locked nucleic acid) (Dominguez, P. L., et al., 2005. Wild-type blocking polymerase chain reaction for detection of single nucleotide minority mutations from clinical specimens Oncogene 24:6830-6834)-mediated PCR clamping technique, COLD-PCR (co-amplification at lower denaturation temperature PCR) (Li, J., et al., 2008. Replacing PCR with COLD-PCR enriches variant DNA sequences and redefines the sensitivity of genetic testing. Nat Med 14:579-584.) and so on.

The REMS-PCR and PNA- or LNA-mediated PCR clamping technique are sensitive and reliable for detection of mutant genes, but the application of these methods has been limited due to its limited applicability and high expense. The recently developed COLD-PCR technique is simple to perform, but has a low amplification factor (3-100×) and a low sensitivity towards minute temperature changes (Li, J., et al., 2008. Replacing PCR with COLD-PCR enriches variant DNA sequences and redefines the sensitivity of genetic testing. Nat Med 14:579-584, Luthra, R., et al., 2009. COLD-PCR finds hot application in mutation analysis. Clin Chem 55:2077-2078).

### SUMMARY OF THE INVENTION

Accordingly, the present inventors have found that a use of a blocking primer together with generic PCR primers allows for the detection of mutant genes with a high sensitivity and specificity, thereby completing the present invention.

The object of the present invention is to provide a composition for detecting mutant genes comprising a forward primer, a reverse primer and a blocking primer.

The forward primer or the reverse primer that is closer to the mutation site comprises a nucleotide sequence complementary to the nucleotide sequence of the mutant gene that excludes the mutation site of the mutant genes in a sample; the blocking primer comprises a nucleotide sequence complementary to the wild-type sequence that corresponds to the mutation site of the mutant genes in the sample; one end of the blocking primer comprises the same nucleotide sequence as the inner end of the primer closer to the mutation site; and the other end of the blocking primer comprises a nucleotide sequence modified by the addition of one or more selected from the group consisting of C3-18 spacers, biotin, di-deoxynucleotide triphosphate, ethylene glycol, amine, and phosphate.

Another object of the present invention is to provide a kit for detecting mutant genes comprising the above composition.

Yet another object of the present invention is to provide a method for detecting mutant genes comprising: performing a polymerase chain reaction (PCR) on a gene sample containing the mutation site to be detected by using a forward primer, a reverse primer and a blocking primer; and identifying a mutation in the PCR product.

Yet another object of the present invention is to provide a method for diagnosing mutation-related diseases using the method for detecting mutant genes.

### BRIEF DESCRIPTION OF THE DRAWINGS

FIG. 1 schematically shows a process of detecting normal DNA and mutant DNA by using generic primers (forward primer and reverse primer) and a blocking primer according to the present invention. The mismatch between the blocking primer and the target mutation site reduces the affinity of blocking primer and thus increases the chance of generic primers annealing to target site, which then enables selective amplification of mutant gene.

FIG. 2 schematically shows the locations of generic primers.

FIG. 3 schematically shows the location of a blocking primer.

FIG. 4 shows the sequence analysis results of PCR products obtained from a PCR reaction on EGFR-mutated DNA diluted with normal DNA at dilution factor of 1:1000 using a blocking primer. Then the results were compared to that of PCR products obtained from PCR that used only generic primers.

FIG. 5 shows the absence of the wild-type peaks in a specimen obtained by diluting mutant DNA with normal DNA in a ratio of 1:1 and 1:10⁻² respectively, and the presence of the heterozygous peaks in a specimen obtained by diluting mutant DNA in a ratio of 1:10⁻⁴.

FIG. 6 shows sensitivity in detection as a function of a distance between the locations of a generic primer and a mutation site (the number of base pairs).

FIG. 7 is a graph showing detection sensitivity as a function of the concentration ratio of blocking primers to generic primers. The x-axis indicates the amount of the blocking primer per 10 pmol of the generic primer. Detection sensitivity was improved with an increase in the amount of the blocking primer, and it reached a plateau when the ratio of blocking primer:generic primer was 5:1 respectively.

FIG. 8 shows detection sensitivity as a function of the melting temperatures (Tm; ° C.) of the wild-type sequence and generic primer duplexes.

FIG. 9 shows detection sensitivity as a function of the melting temperature (Tm; ° C.) of the wild-type sequence and blocking sequence duplexes.

FIG. 10 shows detection sensitivity as a function of the length (bp) of the overlap between the blocking primer and the generic primer.

FIG. 11 shows detection sensitivity as a function of the melting temperature (Tm) of the wild-type sequence and blocking primer duplexes and the annealing temperature of PCR (59° C. in this experiment). It demonstrates that a higher sensitivity was obtained at a temperature higher than the annealing temperature of PCR (59° C. in this experiment), but sensitivity was lost at an extremely high melting temperature (Tm).

FIG. 12 shows that, when the mismatched blocking primer and mutant sequence duplexes have a high melting temperature (T_{m-mismatch}), they are not melted at the annealing temperature of PCR (59° C. in this experiment), resulting in an inferior sensitivity.

FIG. 13 shows the close correlation between detection sensitivity and deviation (ΔTm) between the Tm and T_{m-mismatch} through detection of BRAF V600E, JAK2 V617F and EGFR T790M mutations. Greater ΔTm leads to a higher sensitivity.

FIG. 14 shows the close correlation of ΔTm with detection sensitivity in the detection of KRAS codon 12 mutations. Greater ΔTm indicates higher sensitivity.

FIG. 15 shows that a blocking primer having high melting temperature (Tm) for a wild-type sequence generally has a high sensitivity towards small deletion/insertion mutations.

FIG. 16 shows the suitability of MEMO to quantitative real-time PCR and HRM analysis. Serial dilutions of specimen containing DNA with T790M mutations in EGFR, which were detected through a real-time PCR assay using a DNA-intercalating fluorescence dye show different fluorescence curves depending on the concentrations of the mutant allele.

FIG. 17 shows the standard curves generated by performing quantitative real-time PCR and HRM analysis in quadruplicate. The curves show a linear correlation (r²=0.991) within the range from 1.0×10⁰ to 1.0×10⁻³ (PCR efficiency: 1.45).

FIG. 18 shows the HRM analysis demonstrating that dilutions with a higher concentration of mutant alleles (1.0×10⁰, 1.0×10⁻¹ and 1.0×10⁻²) show a higher melting temperature (84.3-84.4° C.) compared to that of normal samples (83.7° C.), whereas samples with low concentration of mutant allele (<1.0×10⁻³) showed heterozygous melting curves.

FIG. 19 shows the result of amplicon sequencing, which complied with that of HRM analysis (i.e., homozygous mutant peak was apparent in samples with a high concentration of mutant alleles and heterozygous peak was apparent in samples with a low concentration of mutant alleles).

FIG. 20 shows the analysis result of MEMO-PCR with fluorescence primers and fragment analysis identifying a 15-bp deletion in EGFR exon 19 for samples at 1.0×10⁻⁶ dilution.

FIG. 21 shows the analysis result of MEMO-PCR with fluorescence primers and fragment analysis identifying a 4-bp insertion in NPM1 exon 12 for the samples at a 1.0×10⁻⁵ dilution.

FIG. 22 shows an increase in sensitivity of MEMO-PCR and pyrosequencing for the specimens with KRAS mutations. (A) KRAS G12S in 1.0×10⁻², (B) KRAS G12C in 5.0×10⁻³, (C) KRAS G12D 5.0×10², (D) KRAS G12V in 5.0×10⁻³, (E) KRAS G12A in 5.0×10⁻², and (F) KRAS G13D in 2.0×10⁻².

FIG. 23 shows the detection of different BRAF V600E mutations in FNA (fine needle aspirate) samples obtained from thyroid tumor patients by DPO-based ARMS-PCR, conventional sequencing, and MEMO-PCR including sequencing. All three methods detected BRAF V600E mutations in patients having PTC.

FIG. 24 shows detection of different BRAF V600E mutations in FNA (fine needle aspirate) samples from thyroid tumor patients by DPO-based ARMS-PCR, conventional sequencing, and MEMO-PCR including sequencing. For a second patient with PTC, DPO-based ARMS-PCR showed a faint mutant band, and conventional sequencing showed substantially invisible mutant peak, whereas homozygous mutant peak was easily detected by MEMO-PCR.

FIG. 25 shows the detection of different BRAF V600E mutations in FNA (fine needle aspirate) samples obtained from thyroid tumor patients by DPO-based ARMS-PCR, conventional sequencing, and MEMO-PCR including sequencing. ARMS-PCR and conventional sequencing showed negative result and only MEMO-PCR method showed positive result.

FIG. 26 shows the sensitivity achieved by using generic primers that are commonly used for detecting of EGFR, BRAF and JAK mutants and respective blocking primers.

FIG. 27 shows the sensitivity achieved by using generic primers that are used for detecting TP53 mutation and respective blocking primers.

FIG. 28 shows the sensitivity achieved by using generic primers that are commonly used for detecting KRAS mutation and respective blocking primers.

FIG. 29 shows the sensitivity achieved by using generic primers that are used for detecting EGFR and NPM1 deletion/insertion mutations and respective blocking primers.

### DETAILED DESCRIPTION OF THE INVENTION

The present invention has been made in order to develop a method which is capable of effectively detecting tumor-specific mutant DNA present at low concentrations and which is clinically used for the diagnosis of tumors, the determination of a treatment protocol, the detection of residual tumors after treatment, and the like.

The present invention is characterized by providing diagnostic technology having both high sensitivity and high specificity in which mutant DNA is selectively amplified by performing a PCR reaction using a pair of PCR primers and a blocking primer competing with any one of the PCR primers.

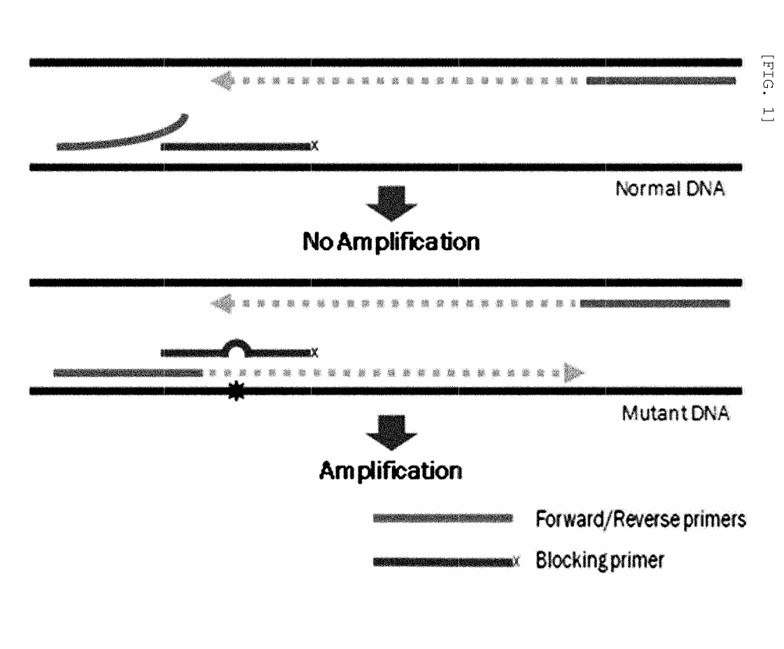

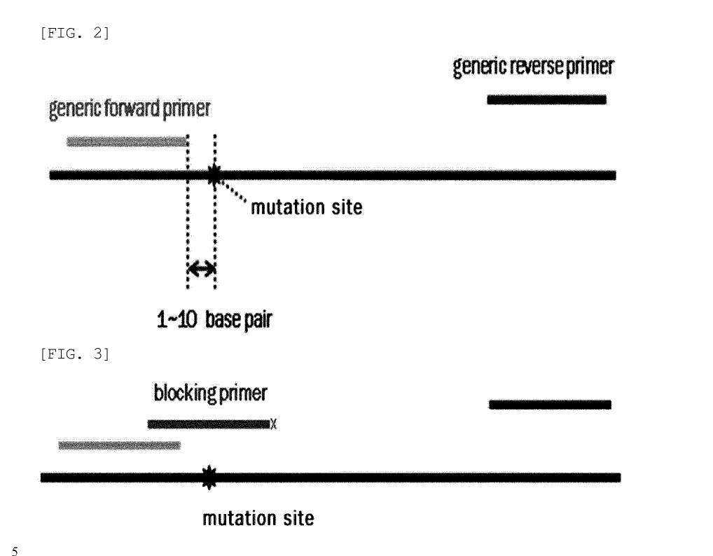

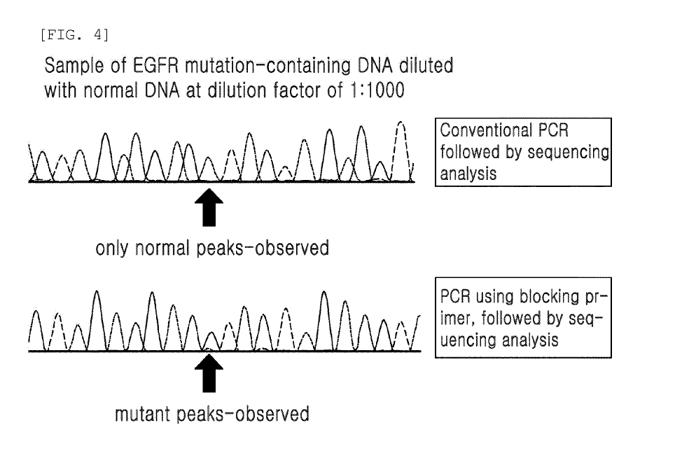

Specifically, a blocking primer strongly binds to wild-type sequences, whereas its affinity for mutant sequences is markedly reduced due to mismatches. The lack of competition by the blocking primer enables selective amplification of mutant sequences by the generic primer pair (FIG. 1).

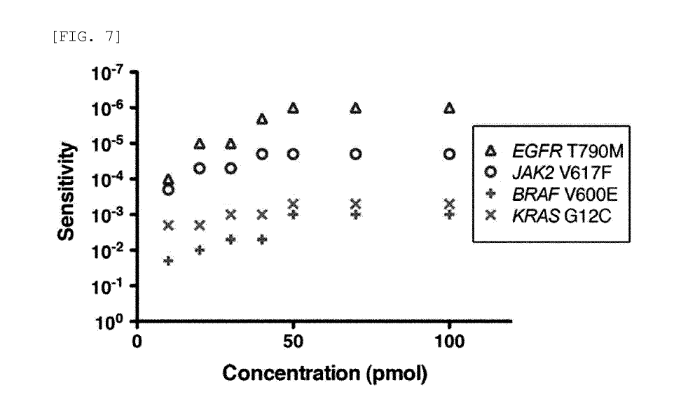

In the present invention, the performance of mutant enrichment with terminal-modified oligonucleotides PCR (MEMO-PCR) was evaluated based on its ability to detect common cancer mutations in the EGFR, KRAS, BRAF, TP53, JAK2, and NPM1 genes. It was observed that a sensitivity of approximately 10⁻¹ to 10⁻⁷ can be achieved by MEMO-PCR in combination with downstream sequencing analysis (FIGS. 6 to 15).

In the present invention, a PCR clamping technique was used to selectively amplify and examine mutation-specific genes. In this method, a blocking primer having a nucleotide sequence complementary to the nucleotide sequence of a wild-type gene is added in a PCR reaction to block the amplification of the wild-type gene. Methods similar thereto, such as REMS-PCR, were developed which use locked nucleic acid (LNA), peptide nucleic acid (PNA) or the like as blocking primers, but such substances are not widely used because they are difficult to prepare and are expensive. In addition, COLD-PCR has the shortcomings of low amplification factor and sensitivity to minute changes in temperature.

Accordingly, the present inventors have conducted studies to solve the above-described problems occurring in the prior art and, as a result, have found that even when one terminal end of generic oligonucleotides is modified, there is no variation in the effect of amplification. Such oligonucleotides modified at one end have an advantage in that the production cost thereof is 10-20 times lower than that of conventional LNA or PNA.

The present invention is directed to a method for detecting mutant DNA present at low concentrations by performing PCR using terminal-modified oligonucleotides and a blocking primer, and the advantages therein of the present invention are that it has efficiency equal to or higher than a method employing LNA or PNA while remaining highly cost-effective. In addition, the method of the present invention has benefits in that it is not sensitive to changes in temperature and enables a mutant gene to be amplified at a higher amplification factor as compared to COLD-PCR.

In addition, according to the present invention, the melting temperature (Tm) and concentration of a blocking primer, the PCR temperature, the overlapping region between a blocking primer and generic primers, and the difference (AT) between Tm and Tm-mismatch have been optimized to increase the efficiency of detection of mutations. This method allows a tumor-specific gene to be detected in a cost-effective and accurate manner compared to conventional detection methods.

Hereinafter, the present invention will be described in detail.

In one aspect, the present invention is directed to a composition for detecting mutant genes, the composition comprising a forward primer, a reverse primer and a blocking primer, wherein the forward primer or the reverse primer that is closer to the mutation site comprises a nucleotide sequence complementary to the nucleotide sequence of the mutant gene that excludes the mutation site of the mutant genes in a sample; wherein the blocking primer comprises a nucleotide sequence complementary to the wild-type sequence that corresponds to the mutation site of the mutant genes in the sample; one end of the blocking primer comprises the same nucleotide sequence as the inner end of the primer closer to the mutation site; and the other end of the blocking primer comprises a nucleotide sequence modified by the addition of one or more selected from the group consisting of C₃₋₁₈ spacers, biotin, di-deoxynucleotide triphosphate, ethylene glycol, amine, and phosphate.

The composition is preferably used to perform a polymerase chain reaction (PCR).

In another aspect, the present invention is directed to a kit for detecting mutant gene, the kit comprising the above composition.

In still another aspect, the present invention is directed to a method for detecting mutant genes, the method comprising the steps of: performing a polymerase chain reaction (PCR) on a gene sample containing the mutation site to be detected by using a forward primer, a reverse primer and a blocking primer; and identifying a mutation in the PCR product, wherein the forward primer or the reverse primer that is closer to the mutation site comprises a nucleotide sequence complementary to the nucleotide sequence of the mutant gene that excludes the mutation site of the mutant genes in a sample; wherein the blocking primer comprises a nucleotide sequence complementary to the wild-type sequence that corresponds to the mutation site of the mutant genes in the sample; one end of the blocking primer comprises the same nucleotide sequence as the inner end of the primer closer to the mutation site; and the other end of the blocking primer comprises a nucleotide sequence modified by the addition of one or more selected from the group consisting of C3-18 spacers, biotin, di-deoxynucleotide triphosphate, ethylene glycol, amine, and phosphate.

In yet another aspect, the present invention is directed to a method for diagnosing mutation-related disease using the above detection method.

As used herein, the term “sample” refers to a gene sample containing the gene mutation site to be detected. Specifically, the sample is meant to include all organism-derived samples in which nuclear and/or mitochondrial genes can be analyzed. The sample may be one selected from cells, tissues, organs, body fluids, and endogenous or exogenous genes (e.g., genes from pathogenic bacteria and/or viruses) extracted therefrom. The cells, tissues, organs, body fluids and the like may be those collected from mammals (e.g., humans, primates, rodents, etc.).

The cells may include the cells of unicellular animals, including viruses or bacteria. For example, for diagnosis of a tumor by way of detection of a gene mutation, the gene sample may be one extracted from the cell of the patient to be diagnosed, and for detection of residual tumors after tumor treatment, the gene sample may be one extracted from the cancer cell of a patient who underwent tumor treatment. For detection of a drug-resistant mutant strain, the gene sample may be extracted from a bacterial or viral strain.

The gene in the sample comprises a target gene (full-length gene) containing the gene mutation site to be detected or a portion of the target gene, which contains the gene mutation site. The portion containing the gene mutation site may be a polynucleotide having a length of approximately 5-1,000,000 bp, preferably approximately 5-100,000 bp, more preferably approximately 5-5000 bp, which contains the gene mutation site.

As used herein, the term “primer” means a short nucleotide sequence, which can form base pairs with a complimentary template and has a free 3′ hydroxyl group which serves as a starting point for the DNA replication of the template. A primer can initiate DNA synthesis in a suitable buffer at a suitable temperature in the presence of polymerization reagents (i.e., DNA polymerases or reverse transcriptases) and four different nucleoside triphosphates. In addition, primers may be sense and antisense nucleotide sequences, each having 7-50 nucleotides, and may be incorporated with additional features without changing their fundamental function of serving as a starting point for DNA synthesis.

As used herein, the term “forward primer and reverse primer” refers to generic primers which are generally used for the amplification of a gene sample containing the gene mutation to be detected. Such forward and reverse primers can be easily determined by those skilled in the art depending on the gene mutation to be detected and the gene containing the mutation.

More specifically, the forward primer and the reverse primer can be designed to include an oligonucleotide having a length of 10-50 bp, preferably 15-35 bp, and an oligonucleotide having a nucleotide sequence complementary thereto and having a length of 10-50 bp, preferably 15-35 bp.

As used herein, the term “wild-type gene” refers to an allele that is most commonly found in nature or is otherwise designated normal. For the purpose of the present invention, the term “wild-type gene” means a normal gene. In the Examples of the present invention, normal genes, including EGFR, BRAF, JAK2, TP53, KRAS and NPM1, were used, but are not limited thereto.

As used herein, the term “mutant gene” refers to a gene that differs from a wild-type gene in DNA structure and sequence or function. In the Examples of the present invention, mutant genes, including EGFR, BRAF, JAK2, TP53, KRAS and NPM1, were used, but are not limited thereto.

As used herein, the expression “primer closer to the mutation site” or “mutation-close primer” refers to one of the forward primer or the reverse primer, which are located closer to the mutation site and designed to have a nucleotide sequence complementary to the nucleotide sequence of the mutant gene, which is at some distance from the mutation site to be detected and has a length of 10-50 bp, preferably 15-35 bp.

The distance between the primer closer to the mutation site and the mutation site is 1-30 bp, preferably 1-20 bp, and more preferably 1-9 bp (FIG. 6).

One of the forward primer or the reverse primer, which are located further from the mutation site may be an oligonucleotide having a length of 10-50 bp, preferably 15-35 bp, which is designed to become a polynucleotide having a length of about 5-1,000,000 bp, preferably about 5-100,000 bp, more preferably 5-5000 bp, by an amplification process.

As used herein, the term “blocking primer” refers to one having the following characteristics:

(A) The blocking primer comprises a nucleotide sequence complementary to the wild-type sequence that corresponds to the mutation site of the mutant genes in the sample. Thus, the blocking primer binds to the wild-type gene such that it interferes with the binding of generic primers to the wild-type gene, thereby blocking the amplification of the wild-type gene. However, the generic primers bind specifically to the mutant gene to amplify the mutant gene. Thus, the blocking primer serves to increase sensitivity and specificity of detection of the mutant gene.

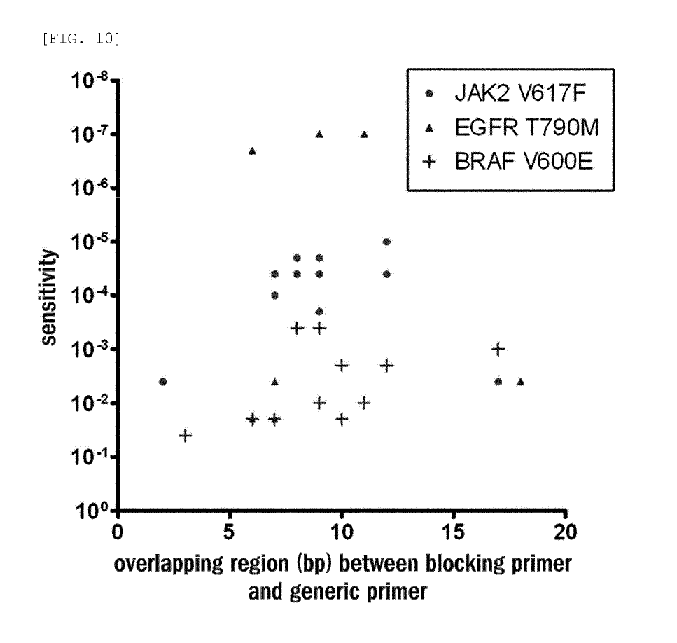

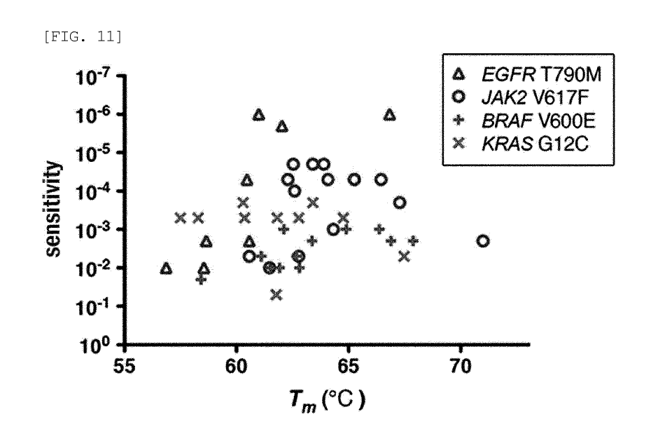

(B) One end of the blocking primer comprises the same nucleotide sequence as the inner end of the primer closer to the mutation site. If the primer closer to the mutation site is the forward primer, said one end of the blocking primer is the 5′ end, and if the primer closer to the mutation site is the reverse primer, said one end of the blocking primer is the 3′ end. The nucleotide sequence having the same nucleotide sequence as the inner end of the primer closer to the mutation site refers to the nucleotide sequence of the region overlapping with the primer closer to the mutation site. The primer closer to the mutation site binds to the mutant gene, so that the nucleotide sequence of the mutant gene is amplified by the inner end of the primer, but one end of the blocking primer in place of the inner end of the primer closer to the mutation site competitively binds to the wild-type gene, such that the wild-type gene cannot be amplified. The length of the nucleotide sequence of the blocking primer that is same as the inner end of the primer closer to the mutation site may be 3 bp or more, for example, 3-50 bp, 3-35 bp, 5-50 bp, or 5-35 bp. Preferably, the length may be 3-13 bp. If the length is less than 3 bp, sufficient sensitivity will not be obtained (FIG. 10).

(C) Also, the other end of the blocking primer is modified so as to block PCR amplification. If the primer closer to the mutation site is the forward primer, said other end is the 3′ end, and if the primer closer to the mutation site is the reverse primer, said other end is the 5′ end. The modification of the end can be performed by attaching to the end of the blocking primer one or more selected from the group consisting of C3-18 spacers (structures consisting of 3-18 consecutive carbon atoms), for example, a C3 spacer (structure consisting of 3 consecutive carbon atoms), a C6 spacer (structure consisting of 6 consecutive carbon atoms), a C12 spacer (structure consisting of 12 consecutive carbon atoms), and a C18 spacer (structure consisting of 18 consecutive carbon atoms), biotin, di-deoxynucleotide triphosphate (ddNTP), ethylene glycol, amine, and phosphate. In the present invention, the 3′ end of the blocking primer was modified by addition of each of a C3 spacer, a phosphate and a C6 amine, and each modification was tested for blocking efficiency, as a result of which these modifications showed similar sensitivities (Example 2). Because the blocking primer was modified at the end, it does not amplify a wild-type gene to which it binds unlike generic primers.

As described above, in the present invention, pair of generic forward and reverse primers and the corresponding blocking primer are competitively reacted with genes, such that the blocking primer preferentially binds to the wild-type gene so that the wild-type gene is not amplified by the forward and reverse primers. On the other hand, one of the forward or reverse primers, which are closer to the mutation site being detected, preferentially binds to the gene having the mutation site such that the mutant gene is normally amplified.

The composition for detecting mutant genes may be used to perform PCR. As used herein, the term “PCR” refers to a process of amplifying a specific target gene to be detected. Examples of polymerase chain reaction (PCR) include a reverse transcriptase polymerase chain reaction (RT-PCR) comprising synthesizing complementary DNA from RNA using reverse transcriptase and performing PCR using the DNA as a template, and real-time PCR comprising amplifying DNA using a fluorescent substance while detecting the amplification product.

In order to achieve different preferences to reactions with a wild-type gene and a mutant gene, the melting temperatures of the primer closer to the mutation site and the blocking primer can be of significance.

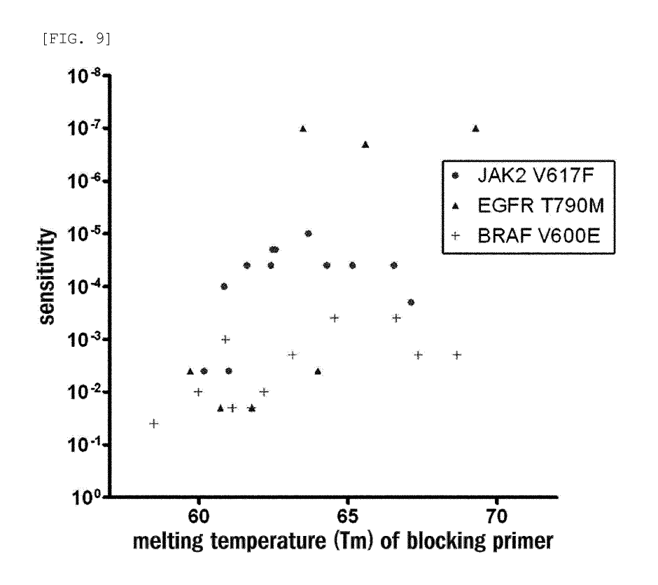

To achieve the desired reactions, the melting temperature (Tm) of the mutation-close primer which is in competition with the blocking primer is 65° C. or lower, preferably 62° C. or lower, for example, 55 to 65° C., or 55 to 62° C., preferably 55 to 62° C., or 58 to 62° C. (FIG. 8), and the melting temperature of the blocking primer is higher than the melting temperature of the primer closer to the mutation site by 0° C. or higher, preferably 2° C. or higher, for example, 0 to 12° C., preferably 2 to 12° C. (FIG. 9).

In addition, the annealing temperature in the PCR reaction of the composition is preferably lower than the melting temperature of the wild type gene/blocking primer duplexes and higher than the melting temperature of the mutant gene/blocking primer duplexes (FIGS. 11 to 15).

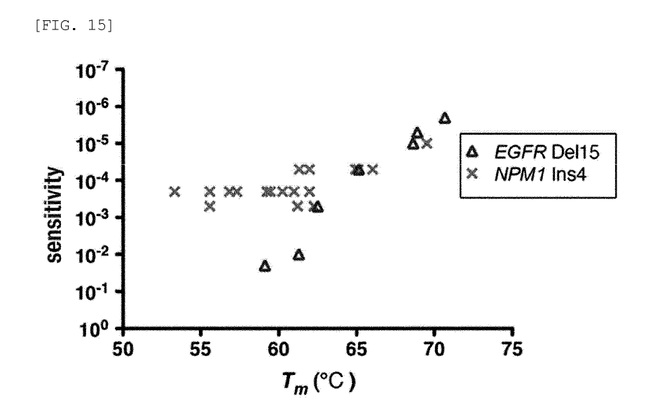

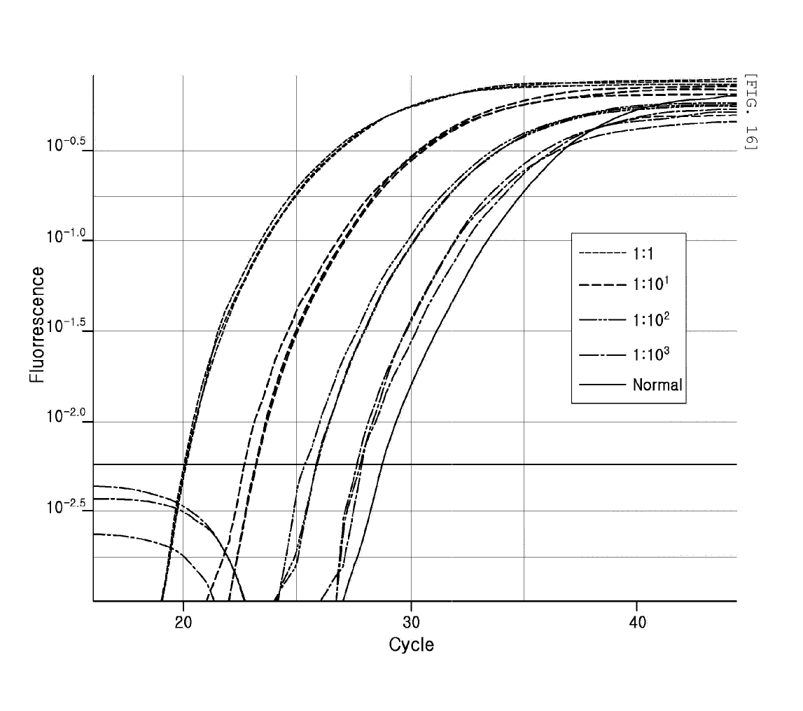

When the melting temperature of the mismatched blocking primer/mutant gene duplexes (Tm-mismatch) is much lower than the melting temperature (Tm) of the wild type sequence/blocking primer duplexes, the affinity of the blocking primer for the mutant sequence is significantly reduced so that the blocking primer becomes less competitive with the generic primer, and thus the binding of the generic primmer is increased to enable the amplification of the mutant sequence, thus providing clear discrimination between the mutant sequence and the normal sequence. This effect can be further increased when the melting temperature of the mismatched blocking primer/mutant sequence duplexes (Tm-mismatch) is lower than the annealing temperature of the PCR reaction (FIGS. 13 and 14).

In addition, PCR conditions are preferably optimized. For example, PCR may be performed under the following conditions:

94° C. for 5 min (1 cycle); and then

50 cycles, each consisting of 30 sec at 94° C., 30 sec at 59° C., and 30 sec at 72° C.; and then

72° C. for 7 min (1 cycle).

The above PCR conditions may be modified in various manners depending on the desired reaction, and such optimal conditions can be easily adopted by those skilled in the art.

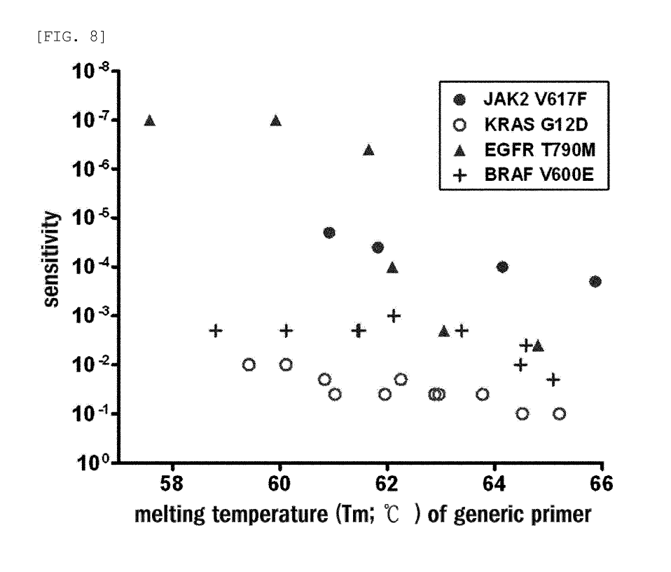

In the composition, the molar concentration ratio between the primer closer to the mutation site and the blocking primer is preferably 1:5 to 1:50. As the concentration of the blocking primer relative to the concentration (mol) of the primer closer to the mutation site increases, sensitivity increases. If the concentration of the blocking primer was about one time higher than that of the primer closer to the mutation site, desired detection efficiency could be obtained, and if it was about 5 times higher than that of the primer close to the mutation site, no significant difference in sensitivity was observed. Thus, the concentration of the blocking primer is preferably 1-5 times higher than that of the primer closer to the mutation site. The upper limit of the concentration of the blocking primer relative to the concentration (mol) of the primer closer to the mutation site is not specifically limited, and the blocking primer may be used in an amount up to about 50 times the amount of the primer closer to the mutation site in view of the economy of the amount of sample used. For example, the concentration of the blocking primer may be 1-50 times, for example, 1-10 times, 5-50 times or 5-10 times, the concentration (mol) of the primer closer to the mutation site (FIG. 7).

The ratio of the concentration (mol) of the forward primer to the reverse primer is not specifically limited and may, for example, be 1:50 to 50:1, preferably 1:10 to 10:1, more preferably 1:5 to 5:1. In view of reaction efficiency and the economy of a sample, the forward primer to the reverse primer is preferably used at a ratio of 1:2 to 2:1, for example, 1:1.

As used herein, the term “mutation” is meant to include all kinds of nuclear and/or mitochondrial gene mutations, including point mutations and small insertion/deletion mutations (e.g., 1-50-bp insertion or deletion mutation). Gene mutations which can be detected by the present invention are not specifically limited and include all kinds of mutations, for example, tumor-specific mutations (useful for diagnosis of tumors), mitochondrial mutations (useful for diagnosis of mitochondrial diseases), or mutations imparting drug resistance to pathogenic bacteria and/or viruses (useful for detection of drug-resistant pathogenic bacterial and/or viral strains present at low concentrations and for diagnosis of pathogenic bacteria and/or virus-related diseases), but are not limited thereto.

The tumor-specific mutation may be a mutation specific to a tumor selected from the group consisting of, for example, various solid cancers, including thyroid cancer, gastric cancer, colorectal cancer, lung cancer, skin cancer, esophageal cancer, oral cancer, pancreatic cancer, bile duct cancer, liver cancer, laryngeal cancer, uterine cancer, ovarian cancer, breast cancer, prostate cancer, brain tumor, neuronal cancer, and bone tumor, myeloproliferative diseases, and blood cancers, including leukemia. In addition, the present invention may also be applied to viral infections, mitochondrial diseases and the like, but is not limited.

The tumor-specific mutation may be a mutation occurring in a gene selected from the group consisting of, for example, KRAS (Kirsten rat sarcoma 2 viral oncogene homolog, NM_{—}004985) gene, APC (Adenomatous polyposis coli; NM_{—}000038), BRAF (Murine sarcoma viral (v-raf) oncogene homolog B1; NM_{—}004333), BRCA1 (Breast cancer-1 gene; NM_{—}007295), BRCA2 (Breast cancer-2, early onset; NM_{—}000059), CDH1 (Cadherin-1 (E-cadherin; uvomorulin); NM_{—}004360), CDKN2A (Cyclin-dependent kinase inhibitor 2A (p16, inhibits CDK4); NM_{—}000077), CTNNB1 (Catenin (cadherin-associated protein), beta 1, 88 kD; NM_{—}001098209), CYLD1 (Cylindromatosis gene; NM_{—}015247), EGFR (Epidermal growth factor receptor; NM_{—}005228), ERBB2 (Avian erythroblastic leukemia viral (v-erb-b2) oncogene homolog 2; NM_{—}004448), FAM123B (Family with sequence similarity 123, member B; NM_{—}152424), FBXW7 (F-box and WD40 domain protein 7; NM_{—}018315), FGFR3 (Fibroblast growth factor receptor-3; NM_{—}022965), FLCN (Folliculin; NM_{—}144606), FLT3 (fms-related tyrosine kinase-3; NM_{—}004119), HRAS (Harvey rat sarcoma viral (v-Ha-ras) oncogene homolog; NM_{—}005343), IDH1 (Isocitrate dehydrogenase, soluble; NM_{—}005896), JAK2 (Janus kinase 2 (a protein-tyrosine kinase); NM_{—}004972), SMCX (Selected cDNA on X, mouse, homolog of; NM_{—}004187), MLH1 (mutL, E. coli, homolog of, 1; NM_{—}000249), MSH2 (mutS, E. coli, homolog of, 2; NM_{—}000251), MSH6 (MutS, E. coli, homolog of, 6; NM_{—}000179), NF1 (Neurofibromin (neurofibromatosis, type I); NM_{—}001128147), NF2 (Merlin; NM_{—}181825), NOTCH1 (Notch, Drosophila, homolog of, 1, translocation-associated; NM_{—}017617), NPM1 (Nucleophosmin 1 (nucleolar phosphoprotein B23, numatrin); NM_{—}001037738), NRAS (Neuroblastoma RAS viral (v-ras) oncogene homolog; NM_{—}002524), NTRK3 (Neurotrophic tyrosine kinase, receptor, type 3; NM_{—}002530), PALB2 (Partner and localizer of BRCA2; NM_{—}024675), PDGFRA (Platelet-derived growth factor receptor, alpha polypeptide; NM_{—}006206), PIK3CA (Phosphatidylinositol 3-kinase, catalytic, alpha polypeptide; NM_{—}006218), PTEN (Phosphatase and tensin homolog (mutated in multiple advanced cancers; NM_{—}000314), RB1 (Retinoblastoma-1; NM_{—}000321), RET (RET transforming sequence; oncogene RET; NM_{—}020630), RUNX1 (Runt-related transcription factor 1 (amll oncogene); NM_{—}001754), SMAD4 (Mothers against decapentaplegic, Drosophila, homolog of, 4; NM_{—}005359), SOCS1 (Suppressor of cytokine signaling 1; NM_{—}003745), STK11 (Serine/threonine protein kinase-11; NM_{—}000455), TP53 (Tumor protein p53; NM_{—}001126116), TSC1 (Hamartin (tuberous sclerosis 1 gene); NM_{—}000368), UTX (Ubiquitously-transcribed TPR gene on X chromosome; NM_{—}021140), and VHL (VHL gene; NM_{—}000551) genes, but is not limited thereto.

More specifically, the tumor-specific mutation may be selected from the group consisting of, for example, EGFR (L858R, T790M and De115), BRAF (V600E), JAK (V617F), TP53 (R175H, R248Q/R248W, R273H/R273c), KRAS (G123/G12C, G12D, G12A, G13D) and NPMI (Ins4), but is not limited thereto.

In addition, the bacterial and/or viral diseases are diseases caused by various bacterial and/or viral infections, and typical examples thereof include hepatitis, cholecystitis, pancreatitis, gastritis, enteritis, cystitis, nephritis, pyelonephritis, dermatitis, myositis, vaginitis, urethritis, prostatitis, pneumonitis, bronchitis, laryngopharyngitis, nasitis, keratitis, iritis, conjunctivitis, otitis media, meningitis, and encephalitis. Typical examples of mutations related to these diseases include a tyrosine-methionine-aspartate-aspartate (YMDD) motif related to lamivudine drug resistance, drug-resistant mutations in hepatitis B virus containing the resistance portion (e.g., point mutations present in codons 528 and 529 in hepatitis B viral genes), or S antigen gene mutations related to vaccination failure, but are not limited thereto. According to the present invention, a virus having the mutation can be effectively detected even when it is present at a very low concentration.

Typical examples of mitochondrial diseases include MELAS (mitochondrial myopathy, encephalopathy, lactic acidosis, and stroke), MERRF (myoclonic epilepsy with ragged red fibers), CPEO (chronic progressive external ophthalmoplegia) and the like, but are not limited thereto. Mutations related to these diseases may be point mutations which are frequently observed in MELAS, MERRF, CPEO and the like, and these mutations are well known in the art.

As used herein, the expression “mutation site of the gene” means a site at which the gene mutation to be detected occurs.

In the present invention, the step of identifying the mutation can be performed by all mutation identification methods which are commonly used in the art, and there is no particular limitation thereon. For example, the mutation can be identified by one or more methods selected from the group consisting of direct sequencing, Taqman probe assay, melting temperature analysis, allele-specific PCR, restriction fragment length polymorphism (RFLP), ARMS (amplification refractory mutation system), ASPCR (allele-specific enzymatic amplification), ASA (allele-specific amplification), PASA (PCR amplification of specific alleles), PAMSA PCR amplification of multiple specific alleles), COP (competitive oligonucleotide priming), E-PCR (enriched PCR), ME-PCR (mutant-enriched PCR), MAMA (mismatch amplification mutation assay), MASA (mutant allele specific amplification), aQRT-PCR (antiprimer quenching-based real-time PCR), REMS-PCR (restriction endonuclease mediated selective PCR), AIRS (artificial introduction of a restriction site), PNA (peptide nucleic acid), LNA (locked nucleic acid), WTB-PCR (wild-type blocking PCR), FLAG (fluorescent amplicon generation), RSM-PCR (restriction site mutation PCR), APRIL-ATM (amplification via primer ligation, at the mutation), PAP (pyrophosphate-activated polymerization), RMC (random mutation capture), CCM (chemical cleavage of mismatches), HRM (high-resolution melting), HET (heteroduplex analysis), SSCP (single-strand conformation polymorphism), DGGE (denaturing gradient gel electrophoresis), CDCE (constant denaturing capillary electrophoresis), dHPLC (denaturing HPLC), iFLP (inverse PCR-based amplified RFLP), COLD-PCR (coamplification at lower denaturation temperature PCR) and the like, but is not limited thereto.

In still another aspect, the present invention provides a method of mutation-related disease, for example, a tumor, mitochondrial disease, or bacterial and/or viral disease, by performing the above method for detection of a gene mutation.

As described above, the gene mutation is specific to tumors or specific to mitochondria or drug-resistant bacteria and/or viruses. Thus, when the above method for detection of a gene mutation is performed on a gene sample obtained from a patient and the gene mutation of interest is identified, the patient can be diagnosed to have a disease related to the gene mutation of interest.

The kind of disease which can be diagnosed by the method for diagnosing a gene mutation-related disease according to the present invention is determined according to the gene mutation to be detected. All kinds of gene mutation-related diseases can be diagnosed by the method of the present invention. For example, a tumor which can be diagnosed by the method of the present invention may be selected from the group consisting of various solid cancers, including thyroid cancer, gastric cancer, colorectal cancer, lung cancer, skin cancer, esophageal cancer, oral cancer, pancreatic cancer, bile duct cancer, liver cancer, laryngeal cancer, uterine cancer, ovarian cancer, breast cancer, prostate cancer, brain tumor, neuronal cancer, and bone tumor, myeloproliferative diseases, and blood cancers, including leukemia; bacterial and/or viral diseases which can be diagnosed by the method of the present invention are diseases caused by various bacterial and/or viral infections and may be selected from the group consisting of, for example, hepatitis, cholecystitis, pancreatitis, gastritis, enteritis, cystitis, nephritis, pyelonephritis, dermatitis, myositis, vaginitis, urethritis, prostatitis, pneumonitis, bronchitis, laryngopharyngitis, nasitis, keratitis, iritis, conjunctivitis, otitis media, meningitis, and encephalitis; and mitochondrial disease which can be diagnosed by the method of the present invention may be selected from the group consisting of MELAS (mitochondrial myopathy, encephalopathy, lactic acidosis, and stroke), MERRF (myoclonic epilepsy with ragged red fibers), CPEO (chronic progressive external ophthalmoplegia) and the like, but the scope of the present invention is not limited thereto.

Patients in which a mutation is to be detected and a tumor is to be diagnosed may be mammals, for example, humans, primates, rodents and the like, and the gene sample may be a total DNA sample separated from the patient, a sample obtained by separating the gene of interest in which the mutation to be detected exists, or a sample comprising a polynucleotide which contains the mutation site of the gene and has a length of about 5-1,000,000 bp, preferably about 5-100,000 bp, more preferably about 5-5000 bp.

Hereinafter, the present invention is described in details with reference to the Examples. However, the Examples are to illustrate the invention only and are not intended to limit the scope of the invention.

### Example 1

### Preparation of DNA Sample

Genomic DNA was extracted from cancer-derived cell lines (including HEL, JAK2 mutant cell line; Mia PaCa, KRAS mutant cell line; H1975, EGFR mutant cell line; SNU-790, BRAF mutant cell line; CCRF-CEM, Kasumi-1, MIA PaCa-2, H1975 and SNU-1196, TP53 mutant cell line; a bone marrow sample obtained from a patient, NPM1 mutant cell line; and all cell lines were purchased from the American Type Culture Collection or the Korean Cell Line Bank, except for an unpublished cell line with an EGFR T790M mutation, which was obtained from the Division of Hematology-Oncology, Department of Medicine at Samsung Medical Center) and from the peripheral blood of a normal person (29 years old healthy woman) using a High Pure PCR Template Preparation Kit (Roche) in the following manner. Each of 200 μl of the extracted samples was added with 200 μl of binding buffer (Roche Diagnostics, Mannheim, Germany) and 40 μl of protease K (Roche Diagnostics). The mixed sample was then incubated at 70° C. for 10 minutes. Then, 100 ji of isopropanol was added to the above sample and mixed thoroughly. Each of the prepared samples was transferred to a collection tube equipped with a High Filter tube (Roche Diagnostics), and was centrifuged at 8000 g for 1 minute.

After centrifugation, the filter tube was separated from the collection tube, and the liquid filtered into the collection tube was discarded. Then the above filter tube was placed in a new collection tube. To this, 500 ji of wash buffer (Roche Diagnostics) was added and the tube was centrifuged at 8000 g for 1 minute. The same procedure for adding wash buffer and centrifuging the sample was repeated once. To remove the remaining wash buffer, the collection tube was centrifuged at the highest centrifugal force for 10 seconds. The filter tube was placed in a new mircotube, and 200 μl of prewarmed elution buffer (Roche Diagnostics) was added thereto, followed by centrifugation at 8000 g for 1 minute. The extracted genomic DNAs were freeze-stored until future testing.

### Example 2

### Construction and 3′ Modification of Blocking Primer

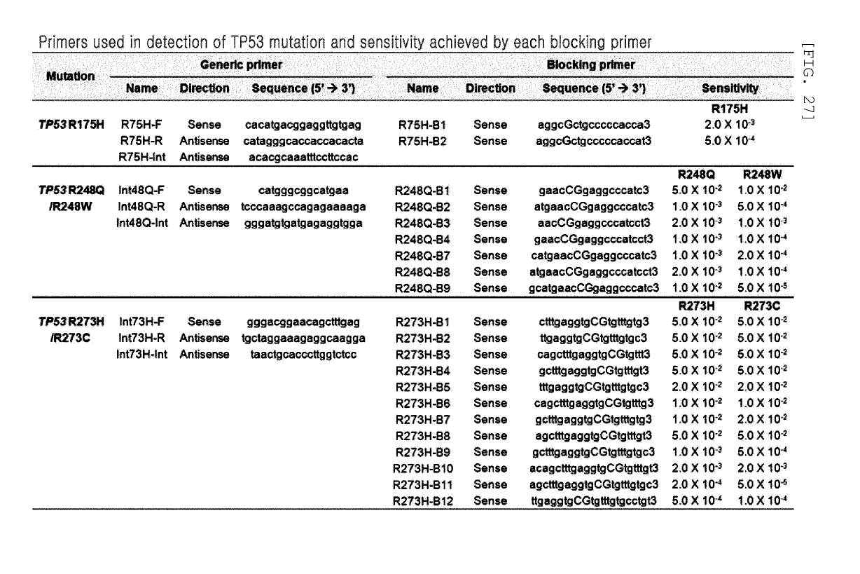

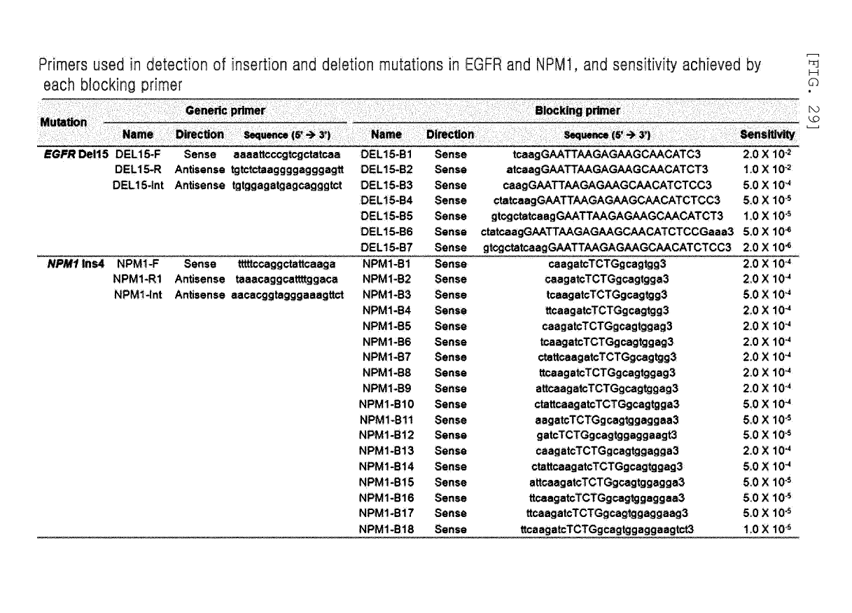

PCR amplification was performed using two generic primers (forward and reverse primers) and one blocking primer designed to encompass the target mutation site and to overlap with one of the generic primers. FIGS. 26 to 29 show generic primers and blocking primers used in the detection of EGFR, BRAF, JAK2, TP53, KRAS, NPM1 gene mutations. The 3′ end of each of the blocking primers was modified by the addition of a C3 spacer, a phosphate or a C6 amine (all from Bioneer, Korea). Each modification was tested for blocking efficiency. No significant differences in sensitivity were observed among the three modifications. Therefore, the C3 spacer modification was used in subsequence experiments.

### Example 3

### Polymerase Chain Reaction (PCR) Amplification

The PCR reaction was performed using the DNA samples prepared in Example 1. The primers used in the PCR are listed in each of the Examples.

The 1 μl of DNA samples prepared in Example 1, 16 μl of sterile distilled water, 1 μl for each of the three primers, and AccuPower PCR Premix (Bioneer, Korea) were mixed together. The PCR was performed using this reaction mixture in the following cycling conditions (hereafter, same cycling conditions were used for detecting other mutations):

[PCR Cycling Conditions]
95° C. for 5 minutes (1 cycle); and then
50 cycles, each consisting of 30 seconds at 94° C., 30 seconds at 59° C., and 30 seconds at 72° C.; and then
72° C. for 7 minutes (1 cycle).

After the amplification reaction, the amplicons were analyzed by electrophoresis to confirm the amplification was successful. First, the amplification products were treated using a Big Dye Terminator Cycle Sequencing Ready Reaction kit (Applied Biosystems) and then sequenced using the ABI Prism 3100 Genetic Analyzer. The results were analyzed using the Sequencher program in comparison to normal nucleotide sequences in order to determine presence of mutation in the DNA sample.

### Example 4

### Experiment for Detection of Mutation

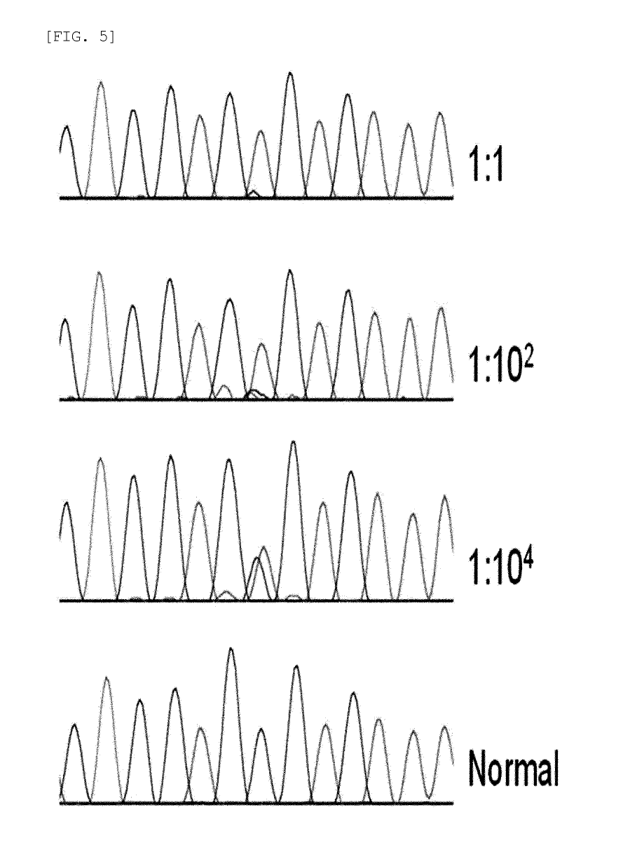

For detecting EGFR T790M mutation, the DNA extracted from the H1975 cancer cell line among the DNA samples prepared in Example 1 was diluted with normal DNA sample (collected under the consent of a donor) at dilution factor of 1:1000. Then the PCR reaction was performed using the diluted DNA sample and the three primers following the same method and cycling condition as in Example 3. FIG. 4 shows the PCR analysis results compared to the PCR results obtained by using generic primers only.

Primers for detection of EGFR T790M mutation-
(SEQ ID NO: 1)

| | |
|---|---|
| Forward primer: | 5′-CACCGTGCAGCTCATCA-3′; |

(SEQ ID NO: 2)

| | |
|---|---|
| Reverse primer: | 5′-cacatatccccatggcaaac-3′; |

(SEQ ID NO: 3

| | |
|---|---|
| Blocking primer: | 5′-GCAGCTCATCACGCAGCTC-3′; |

the 3′ end was modified with a C3 spacer).

The upper side of FIG. 4 shows the sequence analysis results of PCR products obtained by performing PCR reaction of the EGFR mutation-containing DNA sample diluted with normal DNA at a dilution factor of 1:1000 using only generic primers of SEQ ID NO.1 and 2. And the lower side of FIG. 4 shows the sequence analysis results of PCR products obtained by performing PCR reaction of the same using the generic primers of SEQ ID NOS: 1 and 2 together with the blocking primer of SEQ ID NO: 3. At the mutation site of EGFR in FIG. 4, the normal base is cytosine (peak indicated by — — —), and the mutant base is thymidine (peak indicated by —).

As shown in the sequence analysis results in FIG. 4, when the PCR reaction was performed using only the generic primers, only normal cytosine peak was observed in the sample containing mutant DNA, whereas when PCR reaction was performed using the generic primers together with the blocking primer, the mutant peak was clearly observed.

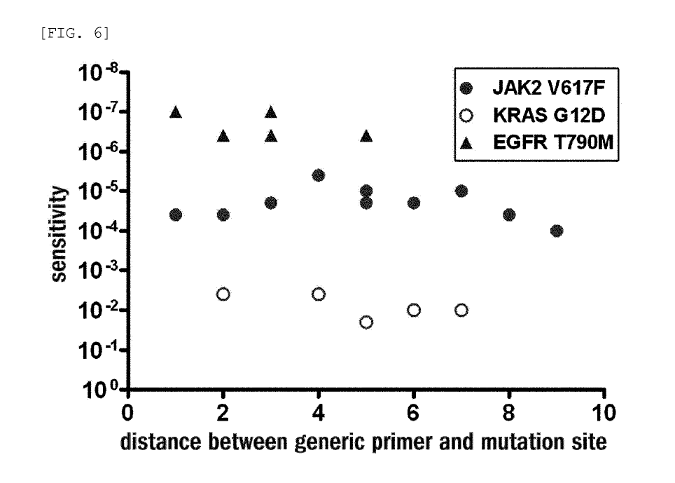

In addition, PCR reaction was performed on the mutant DNA sample which was serially diluted with normal DNA at dilution factor of 1.0×10⁰, 1.0×10⁻¹, 1.0×10⁻² and 1.0×10⁻³ bp using the generic primers of SEQ ID NO.1 and 2 together with the blocking primer of SEQ ID NO. 3. And the sequences of PCR products were analyzed (FIG. 5). According to the sequencing analysis, wild-type peak was absent in the samples diluted at dilution factor of 1.0×10⁰, 1.0×10⁻¹ and 1.0×10⁻². On the other hand, heterozygous peaks were observed in the sample diluted at a factor of 1.0×10⁻⁴. That is, when a ratio of mutant DNA to normal DNA increases, the sensitivity of the method is also improved.

### Example 5

### Investigation of Conditions for Optimal Detection Sensitivity

In order to investigate optimal conditions for obtaining high detection sensitivity, the following experiments were conducted. Here, detection sensitivity was defined as the lowest proportion of mutant DNA that could be consistently detected (>20% of normal peak) in sequencing experiments.

5-1: Experiment on Sensitivity According to Locations of Generic Primers

In order to examine the detection sensitivity as a function of the distance between the locations of generic primers and mutation sites, the following experiment was conducted while varying the distance (bp) between the location of generic primers and mutation site.

To be more specific, in order to examine detection sensitivity as a function of the distance (bp) between the location of JAK2 V617F, KRAS G12D or EGFR T790M mutation and the location of generic primers, PCR reaction was performed using the primers shown in Tables 1 to 3 following the method and conditions described in Example 3. Then the PCR products were analyzed by sequencing. The examined detection sensitivity is shown in Tables 1 to 3 and FIG. 6.

TABLE 1
Analysis of JAK2 V617F mutation

| cells and primers used | | | |
|---|---|---|---|
| tumor cell line | | | detection |
| used | | HEL | sensitivity |
| forward | 1 | GCATTTGGTTTTAAATTATGGAGTATGT | 0.00004 |
| primer | | (SEQ ID NO: 4) | |
| (number of | 2 | CAAGCATTTGGTTTTAAATTATG | 0.00004 |
| base pairs | | (SEQ ID NO: 5) | |
| from | 3 | GCATTTGGTTTTAAATTATGGAGTAT | 0.00002 |
| mutation | | (SEQ ID NO: 6) | |
| site) | 4 | AGCATTTGGTTTTAAATTATGGAGTA | 0.000004 |
| | | (SEQ ID NO: 7) | |
| | 5 | AAGCATTTGGTTTTAAATTATGGAGT | 0.00001 |
| | | (SEQ ID NO: 8) | |
| | 5 | AGCATTTGGTTTTAAATTATGGAGT | 0.00002 |
| | | (SEQ ID NO: 9) | |
| | 6 | AAGCATTTGGTTTTAAATTATGGAG | 0.00002 |
| | | (SEQ ID NO: 10) | |
| | 7 | CAAGCATTTGGTTTTAAATTATGGA | 0.00001 |
| | | (SEQ ID NO: 11) | |
| | 8 | ACAAGCATTTGGTTTTAAATTATGG | 0.00004 |
| | | (SEQ ID NO: 12) | |
| | 9 | CACAAGCATTTGGTTTTAAATTATG | 0.0001 |
| | | (SEQ ID NO: 13) | |

| | |
|---|---|
| reverse primer | tgaaaaggccagttattccaa |
| | (SEQ ID NO: 14) |
| blocking primer | GGAGTATGTGTCTGTGGAGACGAG |
| | (SEQ ID NO: 15) |

TABLE 2
Analysis of KRAS G12D mutation

| cells and primers used | | | |
|---|---|---|---|
| tumor cell line | | | detection |
| used | | HIT-T15 | sensitivity |
| forward | 2 | CTGAATATAAACTTGTGGTAGTTGGAG | 0.004 |
| primer | | (SEQ ID NO: 16) | |
| (number of | 4 | ACTGAATATAAACTTGTGGTAGTTGGA | 0.004 |
| base pairs | | (SEQ ID NO: 17) | |
| from | 5 | GACTGAATATAAACTTGTGGTAGTTGG | 0.02 |
| mutation | | (SEQ ID NO: 18) | |
| site) | 6 | aATGACTGAATATAAACTTGTGGTAGTTG | 0.01 |
| | | (SEQ ID NO: 19) | |
| | 7 | aaaATGACTGAATATAAACTTGTGGTAGTT | 0.01 |
| | | (SEQ ID NO: 20) | |

| | |
|---|---|
| reverse primer | ttgaaacccaaggtacatttca |
| | (SEQ ID NO: 21) |
| blocking primer | TAGTTGGAGCTGGTGGCGTAG |
| | (SEQ ID NO: 22) |

TABLE 3
Analysis of EGFR T790M mutation

| cells and primers used | | | |
|---|---|---|---|
| tumor cell line | | | detection |
| used | | H1975 | sensitivity |
| forward | 1 | CACCGTGCAGCTCACAC | 0.0000001 |
| primer | | (SEQ ID NO: 23) | |
| (number of | 2 | CACCGTGCAGCTCATCA | 0.0000002 |
| base pairs | | (SEQ ID NO: 24) | |
| from | 3 | CCACCGTGCAGCTCATC | 0.0000002 |
| mutation | | (SEQ ID NO: 25) | |
| site) | 4 | TCCACCGTGCAGCTCAT | 0.0000002 |
| | | (SEQ ID NO: 26) | |
| | 6 | CCTCCACCGTGCAGCTC | 0.0000001 |
| | | (SEQ ID NO: 27) | |

| | |
|---|---|
| reverse primer | cacatatccccatggcaaac |
| | (SEQ ID NO: 28) |
| blocking primer | GCAGCTCATCACGCAGCTC |
| | (SEQ ID NO: 29) |

As shown in Tables 1 to 3 and FIG. 6, the optimal distance between the location of the generic primer (forward primer) and the mutation site was in a range of 1 to 9 bp, and no significant difference in sensitivity was observed within this range.

5-2: Experiment on Detection Sensitivity According to the Ratio of Concentrations of Generic Primer to Blocking Primer

In order to examine detection sensitivity as a function of the concentration ratio of a generic primer to a blocking primer, detection sensitivity was measured using the following four sets of primers according to the method in Example 3. And the molar concentration of the blocking primer relative to the generic primer closer to the mutation was changed in the range from 1 to 10.

Primers for detection of JAK2 V617F mutation -
Forward primer:
(SEQ ID NO: 5)
5′-CAAGCATTTGGTTTTAAATTATGG-3′;
Reverse primer:
(SEQ ID NO: 14)
5′-tgaaaaggccagttattccaa-3′;
Blocking primer:
(SEQ ID NO: 15
5′-GGAGTATGTGTCTGTGGAGACGAG-3′;
the 3′ end was modified with a C3 spacer).
Primers for detection of KRAS G12D mutation -
Forward primer:
(SEQ ID NO: 16)
5′-CTGAATATAAACTTGTGGTAGTTGGAG-3′;
Reverse primer:
(SEQ ID NO: 21)
5′-ttgaaacccaaggtacatttca-3′;
Blocking primer:
(SEQ ID NO: 22
5′-TAGTTGGAGCTGGTGGCGTAG-3′;
the 3′ end was modified with a C3 spacer).
Primers for detection of EGFR T790M mutation -
Forward primer:
(SEQ ID NO: 23)
5′-CACCGTGCAGCTCATCA-3′;
Reverse primer:
(SEQ ID NO: 28)
5′-cacatatccccatggcaaac-3′;
Blocking primer:
(SEQ ID NO: 29
5′-GCAGCTCATCACGCAGCTC-3′;
the 3′ end was modified with a C3 spacer).
Primers for detection of BRAF V600E mutation -
Forward primer:
(SEQ ID NO: 55)
5′- cagtaaaaataggtgattttggtctagc-3′;
Reverse primer:
(SEQ ID NO: 61)
5′- ctgatttttgtgaatactgggaact -3′;
Blocking primer:
(SEQ ID NO: 93
5′-ggtgattttggtctagctacagTga3-3′;
the 3′ end was modified with a C3 spacer).

The results are shown in FIG. 7. As shown in FIG. 7, when the concentration ratio of the blocking primer relative to the generic primer increased, the sensitivity was also increased. When the concentration of the blocking primer was more than 5 times higher than that of the generic primer, no significant differences in sensitivity were observed. Thus, it is evident that the best results can be obtained when the blocking primers are added at 5 times or more of the concentration relative to the generic primers.

5-3: Experiment on Detection Sensitivity According to Melting Temperature of Generic Primers

In order to examine detection sensitivity as a function of the melting temperature of generic primers, detection sensitivity was measured using the following three sets of primers according to the method of Example 3 while varying the melting temperature (Tm, ° C.) of the generic forward primer (primer closer to the mutation) in a range from 58 to 66° C. The cancer cell lines and primers used are summarized in Tables 4 to 7, and the obtained results are shown in FIG. 8.

TABLE 4
Analysis of JAK2 V617F mutation

| cells and primers used | | | |
|---|---|---|---|
| tumor cell line | | | detection |
| used | | HEL | sensitivity |
| forward | 60.92 | AGCATTTGGTTTTAAATTATGGAGTATG | 0.00002 |
| primer | | (SEQ ID NO: 30) | |
| (melting | 61.82 | AAGCATTTGGTTTTAAATTATGGAGTATG | 0.00004 |
| temperature; | | (SEQ ID NO: 31) | |
| ° C.) | 64.14 | ACAAGCATTTGGTTTTAAATTATGGAGTATG | 0.0001 |
| | | (SEQ ID NO: 32) | |
| | 65.87 | CACAAGCATTTGGTTTTAAATTATGGAGTATG | 0.0002 |
| | | (SEQ ID NO: 33) | |

| | |
|---|---|
| reverse primer | tgaaaaggccagttattccaa |
| | (SEQ ID NO: 14) |
| blocking primer | GGAGTATGTGTCTGTGGAGACGAG |
| | (SEQ ID NO: 15) |

TABLE 5
Analysis of KRAS G12D mutation

| cells and primers used | | | |
|---|---|---|---|
| tumor cell line | | | detection |
| used | | HIT-T15 | sensitivity |
| forward | 59.42 | ACTGAATATAAACTTGTGGTAGTTGGAG | 0.01 |
| primer | | (SEQ ID NO: 34) | |
| (melting | 60.11 | GACTGAATATAAACTTGTGGTAGTTGGA | 0.01 |
| temperature; | | (SEQ ID NO: 35) | |
| ° C.) | 60.83 | GACTGAATATAAACTTGTGGTAGTTGGAG | 0.02 |
| | | (SEQ ID NO: 36) | |
| | 61.02 | ATGACTGAATATAAACTTGTGGTAGTTGG | 0.04 |
| | | (SEQ ID NO: 37) | |
| | 61.95 | aATGACTGAATATAAACTTGTGGTAGTTGG | 0.04 |
| | | (SEQ ID NO: 38) | |
| | 62.25 | TGACTGAATATAAACTTGTGGTAGTTGGA | 0.02 |
| | | (SEQ ID NO: 39) | |
| | 62.88 | TGACTGAATATAAACTTGTGGTAGTTGGAG | 0.04 |
| | | (SEQ ID NO: 40) | |
| | 62.96 | ATGACTGAATATAAACTTGTGGTAGTTGGAG | 0.04 |
| | | (SEQ ID NO: 41) | |
| | 63.77 | aATGACTGAATATAAACTTGTGGTAGTTGGAG | 0.04 |
| | | (SEQ ID NO: 42) | |
| | 64.51 | aaATGACTGAATATAAACTTGTGGTAGTTGGAG | 0.1 |
| | | (SEQ ID NO: 43) | |
| | 65.2 | aaaATGACTGAATATAAACTTGTGGTAGTTGGAG | 0.1 |
| | | (SEQ ID NO: 44) | |

| | |
|---|---|
| reverse primer | ttgaaacccaaggtacatttca |
| | (SEQ ID NO: 21) |
| blocking primer | TAGTTGGAGCTGGTGGCGTAG |
| | (SEQ ID NO: 22) |

TABLE 6
Analysis of EGFR T790M mutation

| cells and primers used | | | |
|---|---|---|---|
| tumor cell line | | | detection |
| used | | H1975 | sensitivity |
| forward | 57.57 | CCACCGTGCAGCTCAT | 0.0000001 |
| primer | | (SEQ ID NO: 45) | |
| (melting | 59.92 | TCCACCGTGCAGCTCAT | 0.0000001 |
| temperature; | | (SEQ ID NO: 46) | |
| ° C.) | 59.92 | CCACCGTGCAGCTCATC | 0.0000001 |
| | | (SEQ ID NO: 47) | |
| | 61.65 | CCTCCACCGTGCAGCTC | 0.0000004 |
| | | (SEQ ID NO: 48) | |
| | 62.09 | TCCACCGTGCAGCTCATC | 0.0001 |
| | | (SEQ ID NO: 49) | |
| | 63.05 | CTCCACCGTGCAGCTCATC | 0.002 |
| | | (SEQ ID NO: 50) | |
| | 64.8 | CCTCCACCGTGCAGCTCAT | 0.004 |
| | | (SEQ ID NO: 51) | |

| | |
|---|---|
| reverse primer | cacatatccccatggcaaac |
| | (SEQ ID NO: 28) |
| blocking primer | GCAGCTCATCACGCAGCTC |
| | (SEQ ID NO: 29) |

TABLE 7
Analysis of BRAF V600E mutation

| cells and primers used | | | |
|---|---|---|---|
| tumor cell line | | | detection |
| used | | SNU790 | sensitivity |
| forward | 58.8 | AGTAAAAATAGGTGATTTTGGTCTAGC | 0.002 |
| primer | | (SEQ ID NO: 52) | |
| (melting | 60.11 | CACAGTAAAAATAGGTGATTTTGGTCTA | 0.002 |
| temperature; | | (SEQ ID NO: 53) | |
| ° C.) | 61.45 | ACAGTAAAAATAGGTGATTTTGGTCTAGC | 0.002 |
| | | (SEQ ID NO: 54) | |
| | 61.48 | TCACAGTAAAAATAGGTGATTTTGGTCTA | 0.002 |
| | | (SEQ ID NO: 55) | |
| | 62.12 | CTCACAGTAAAAATAGGTGATTTTGGTCTA | 0.001 |
| | | (SEQ ID NO: 56) | |
| | 63.38 | CACAGTAAAAATAGGTGATTTTGGTCTAGC | 0.002 |
| | | (SEQ ID NO: 57) | |
| | 64.48 | CCTCACAGTAAAAATAGGTGATTTTGGTCTA | 0.01 |
| | | (SEQ ID NO: 58) | |
| | 64.58 | TCACAGTAAAAATAGGTGATTTTGGTCTAGC | 0.004 |
| | | (SEQ ID NO: 59) | |
| | 65.08 | CTCACAGTAAAAATAGGTGATTTTGGTCTAGC | 0.02 |
| | | (SEQ ID NO: 60) | |

| | |
|---|---|
| reverse primer | ctgatttttgtgaatactgggaact |
| | (SEQ ID NO: 61) |
| blocking primer | TGGTCTAGCTACAGTGAAATCTCGATGG |
| | (SEQ ID NO: 88) |

5-4: Experiment on Detection Sensitivity According to Melting Temperature of Blocking Primers

In order to examine detection sensitivity as a function of the melting temperature of blocking primers, detection sensitivity was measured using the following three sets of primers according to the method of Example 3 while varying the melting temperature of the blocking primers in a range from 58 to 70° C. The tumor cell lines and primers used are summarized in Tables 8 to 10, and the obtained results are shown in FIG. 9.

TABLE 8
Analysis of JAK2 V617F mutation

| cells and primers used | | | |
|---|---|---|---|
| tumor cell line | | | detection |
| used | | HEL | sensitivity |
| blocking | 60.07 | TTAAATTATGGAGTATGTGTCTGTGGA | 0.004 |
| primer | | (SEQ ID NO: 62) | |
| (melting | 60.9 | TGTGTCTGTGGAGACGAGAgtaag | 0.004 |
| temperature; | | (SEQ ID NO: 63) | |
| ° C.) | 67.01 | TGGAGTATGTGTCTGTGGAGACGAGAg | 0.0002 |
| | | (SEQ ID NO: 64) | |
| | 60.74 | GAGTATGTGTCTGTGGAGACGAGA | 0.0001 |
| | | (SEQ ID NO: 65) | |
| | 61.51 | GGAGTATGTGTCTGTGGAGACGA | 0.00004 |
| | | (SEQ ID NO: 66) | |
| | 63.57 | TTATGGAGTATGTGTCTGTGGAGACG | 0.00001 |
| | | (SEQ ID NO: 67) | |
| | 65.05 | TTATGGAGTATGTGTCTGTGGAGACGA | 0.00004 |
| | | (SEQ ID NO: 68) | |
| | 62.46 | TGGAGTATGTGTCTGTGGAGACG | 0.00002 |
| | | (SEQ ID NO: 69) | |
| | 62.37 | GGAGTATGTGTCTGTGGAGACGAG | 0.00002 |
| | | (SEQ ID NO: 70) | |
| | 62.31 | GAGTATGTGTCTGTGGAGACGAGAgt | 0.00004 |
| | | (SEQ ID NO: 71) | |
| | 64.19 | TGGAGTATGTGTCTGTGGAGACGA | 0.00004 |
| | | (SEQ ID NO: 72) | |
| | 66.44 | TGGAGTATGTGTCTGTGGAGACGAGA | 0.00004 |
| | | (SEQ ID NO: 73) | |

| | |
|---|---|
| forward primer | CAAGCATTTGGTTTTAAATTATGG |
| | (SEQ ID NO: 5) |
| reverse primer | tgaaaaggccagttattccaa |
| | (SEQ ID NO: 14) |

TABLE 9
Analysis of EGFR T790M mutation

| cells and primers used | | | |
|---|---|---|---|
| tumor cell line | | | detection |
| used | | H1975 | sensitivity |
| blocking | 59.7 | AGCTCATCACGCAGCTCAT | 0.004 |
| primer | | (SEQ ID NO: 74) | |
| (melting | 63.99 | CCACCGTGCAGCTCATCAC | 0.004 |
| tempera- | | (SEQ ID NO: 75) | |
| ture; ° C.) | 61.77 | CTCATCACGCAGCTCATGC | 0.02 |
| | | (SEQ ID NO: 76) | |
| | 60.72 | TCATCACGCAGCTCATGC | 0.02 |
| | | (SEQ ID NO: 77) | |
| | 63.49 | GCAGCTCATCACGCAGCTC | 0.0000001 |
| | | (SEQ ID NO: 78) | |
| | 65.59 | GCTCATCACGCAGCTCATGC | 0.0000002 |
| | | (SEQ ID NO: 79) | |
| | 69.29 | GTGCAGCTCATCACGCAGCTCAT | 0.0000001 |
| | | (SEQ ID NO: 80) | |

| | |
|---|---|
| forward primer | CACCGTGCAGCTCATCA |
| | (SEQ ID NO: 1) |
| reverse primer | cacatatccccatggcaaac |
| | (SEQ ID NO: 2) |

TABLE 10
Analysis of BRAF V600E mutation

| cells and primers used | | | |
|---|---|---|---|
| tumor cell line | | | detection |
| used | | SNU790 | sensitivity |
| blocking | 58.49 | AGCTACAGTGAAATCTCGATGG | 0.04 |
| primer | | (SEQ ID NO: 81) | |
| (melting | 59.97 | TGGTCTAGCTACAGTGAAATCTCG | 0.01 |
| temperature; | | (SEQ ID NO: 82) | |
| ° C.) | 60.9 | GGTGATTTTGGTCTAGCTACAGTGA | 0.001 |
| | | (SEQ ID NO: 83) | |
| | 62.18 | TTTGGTCTAGCTACAGTGAAATCTCG | 0.01 |
| | | (SEQ ID NO: 84) | |
| | 63.14 | TTTTGGTCTAGCTACAGTGAAATCTCG | 0.002 |
| | | (SEQ ID NO: 85) | |
| | 64.54 | GGTCTAGCTACAGTGAAATCTCGATGG | 0.0004 |
| | | (SEQ ID NO: 86) | |
| | 66.62 | TGGTCTAGCTACAGTGAAATCTCGATGG | 0.0004 |
| | | (SEQ ID NO: 87) | |
| | 67.36 | TTGGTCTAGCTACAGTGAAATCTCGATGG | 0.002 |
| | | (SEQ ID NO: 88) | |
| | 68.65 | TTTTGGTCTAGCTACAGTGAAATCTCGATGG | 0.002 |
| | | (SEQ ID NO: 89) | |
| | 61.13 | TTGGTCTAGCTACAGTGAAATCTCG | 0.02 |
| | | (SEQ ID NO: 90) | |
| | 61.1 | TCTAGCTACAGTGAAATCTCGATGG | 0.02 |
| | | (SEQ ID NO: 91) | |
| | 61.76 | GTCTAGCTACAGTGAAATCTCGATGG | 0.02 |
| | | (SEQ ID NO: 92) | |

| | |
|---|---|
| forward primer | ctgatttttgtgaatactgggaact |
| | (SEQ ID NO: 61) |
| reverse primer | CACAGTAAAAATAGGTGATTTTGGTCTA |
| | (SEQ ID NO: 54) |

As shown in FIG. 9, as the melting temperature (Tm) of the blocking primer increased, the sensitivity also increased. In addition, the best results were obtained when the melting temperature (Tm) of the blocking primer was at least 2° C. higher than the melting temperature of the generic primer shown in FIG. 8.

5-5: Experiment on Detection Sensitivity According to Melting Temperature (Tm) and the Melting Temperature of Mismatched Blocking Primer/Mutant Sequence Duplexes (T_{m-mismatch})

When the melting temperature of the mismatched blocking primer and mutant sequence duplexes (T_{m-mismatch}) is much lower than the melting temperature (Tm) of the normal sequence and blocking primer duplexes, the affinity of the blocking primer for the mutant sequence is significantly reduced. This results in the loss of competition of the blocking primer. Therefore the binding of the generic primmer is increased enabling the amplification of mutant sequences, which then increases the ability to distinguish the mutant sequence from the normal sequence. The present invention studied the correlation of sensitivity with the difference (ΔTm) between the melting temperature of the normal sequence and blocking primer duplexes and the melting temperature of the mismatched blocking primer and mutant sequence duplexes. As a result, mutations with low ΔTm (e.g., BRAF V600E mutation) showed moderate degrees of sensitivity, whereas mutations with high ΔTm (e.g., EGFR T790M mutation) showed a high sensitivity. This feature was more obvious for the various mutations in KRAS codons 12 and 13 (FIGS. 13 and 14). For small deletion and insertion mutations, blocking primers with a higher Tm generally showed increased sensitivities (FIG. 15).

The sensitivity can be increased when the melting temperature of the mismatched blocking primer/mutant sequence duplexes (T_{m-mismatch}) is lower than the annealing temperature of PCR. The mismatched blocking primer/mutant sequence duplexes should be dissociated during the annealing step in PCR, but when the melting temperature of the mismatched blocking primer/mutant sequence duplexes (T_{m-mismatch}) is higher than the annealing temperature of PCR, the blocking primer is still bound to the mutant DNA sequence even at the annealing temperature, hindering the binding of the generic primers. For this reason, the melting temperature of the mutant sequence and blocking primer duplexes needs to be lower than the annealing temperature of PCR.

As shown in the results of the experiment, sensitivity was increased when the melting temperature (Tm) of the normal sequence/blocking primer duplexes was higher than the annealing temperature of PCR (59° C.). However, the sensitivity was reduced again at an extremely high melting temperature (Tm) (FIG. 11).

The present inventors calculated the melting temperature of the mismatched blocking primer/mutant sequence duplexes (T_{m-mismatch}) using the neighbor joining algorithm of SantaLucia et al.

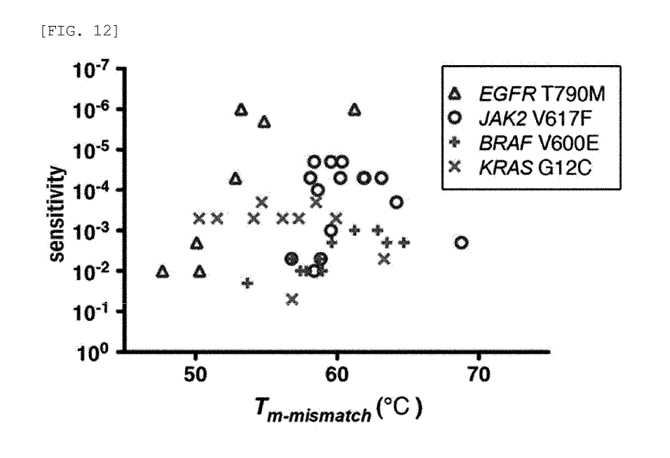

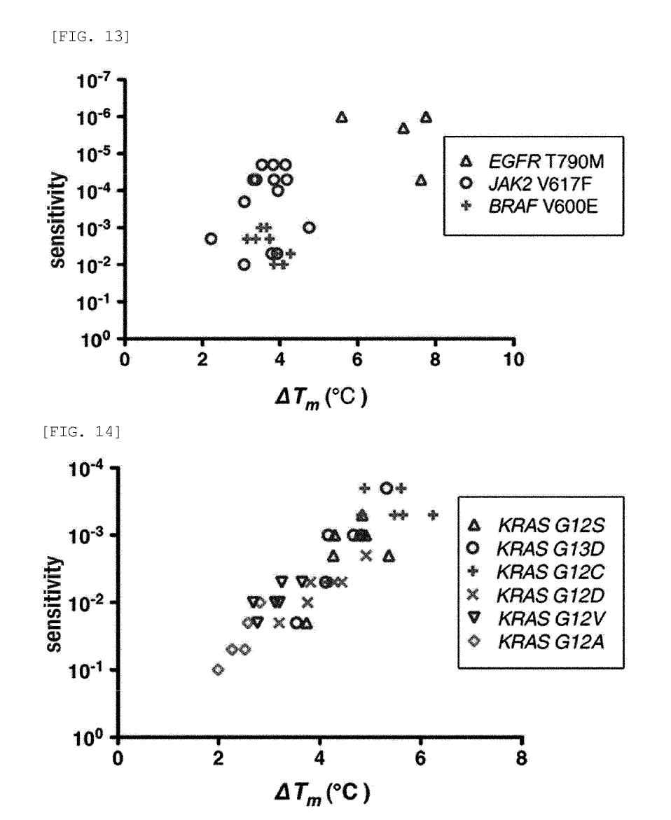

When the melting temperature of the mismatched blocking primer/mutant sequence duplexes (T_{m-mismatch}) was higher than the annealing temperature of PCR (60° C.), sensitivity was reduced (FIG. 12). Table 11 shows the highest sensitivities achieved by MEMO-PCR and downstream sequencing using various sets of primers.

TABLE 11
Cancer mutations evaluated and the highest sensitivities
achieved through MEMO-PCR and sequencing

| | | sample | highest |
|---|---|---|---|
| gene | mutation | (cell line) | sensitivities |
| EGFR | L858R | H1975 | 1.0 × 10⁻³ |
| | T790M | UC^{b} | 1.0 × 10⁻⁶ |
| | Exon 19 Del15 | PC9 | 2.0 × 10⁻⁶ |
| BRAF | V600E | SNU-790 | 1.0 × 10⁻³ |
| TP53 | R175H | CCRF-CEM | 5.0 × 10⁻⁴ |
| | R248Q | Kasumi-1 | 1.0 × 10⁻³ |
| | R248W | MIA PaCa-2 | 5.0 × 10⁻⁵ |
| | R273H | H1975 | 2.0 × 10⁻⁴ |
| | R273C | SNU-1196 | 5.0 × 10⁻⁵ |
| KRAS | G12S | A549 | 5.0 × 10⁻⁴ |
| | G12C | MIA PaCa-2 | 2.0 × 10⁻⁴ |
| | G12D | CCRF-CEM | 5.0 × 10⁻⁴ |
| | G12V | Capan-1 | 2.0 × 10⁻³ |
| | G12A | SW1116 | 2.0 × 10⁻³ |
| | G13D | DLD-1 | 2.0 × 10⁻⁴ |
| JAK2 | V617F | HEL | 2.0 × 10⁻⁵ |
| NPM1 | Exon 12 Ins4 | Patient sample | 1.0 × 10⁻⁵ |

^{a}Best sensitivity that could be obtained upon testing different sets of primers
^{b}Unpublished cell line

5-6: Experiment on Detection Sensitivity According to Overlapping Region Between Blocking Primer and Generic Primer

In order to examine the detection sensitivity as a function of the length (number of base pairs) of the overlapping region between the blocking primer and the generic primer, the detection sensitivity was measured using the following 3 sets of primers according to the method of Example 2. The tumor cell lines and primers used for the experiments are summarized in Tables 12 to 14 below, and the obtained results are shown in FIG. 10.

TABLE 12
Analysis of JAK2 V617 mutation

| cells and primers used | | | |
|---|---|---|---|
| tumor cell line | | | detection |
| used | | HEL | sensitivity |
| blocking | 17 | TTAAATTATGGAGTATGTGTCTGTGGA | 0.004 |
| primer | | (SEQ ID NO: 62) | |
| (overlapping | 2 | TGTGTCTGTGGAGACGAGAgtaag | 0.004 |
| region, bp) | | (SEQ ID NO: 63) | |
| | 9 | TGGAGTATGTGTCTGTGGAGACGAGAg | 0.0002 |
| | | (SEQ ID NO: 64) | |
| | 7 | GAGTATGTGTCTGTGGAGACGAGA | 0.0001 |
| | | (SEQ ID NO: 65) | |
| | 8 | GGAGTATGTGTCTGTGGAGACGA | 0.00004 |
| | | (SEQ ID NO: 66) | |
| | 12 | TTATGGAGTATGTGTCTGTGGAGACG | 0.00001 |
| | | (SEQ ID NO: 67) | |
| | 12 | TTATGGAGTATGTGTCTGTGGAGACGA | 0.00004 |
| | | (SEQ ID NO: 68) | |
| | 9 | TGGAGTATGTGTCTGTGGAGACG | 0.00002 |
| | | (SEQ ID NO: 69) | |
| | 8 | GGAGTATGTGTCTGTGGAGACGAG | 0.00002 |
| | | (SEQ ID NO: 70) | |
| | 7 | GAGTATGTGTCTGTGGAGACGAGAgt | 0.00004 |
| | | (SEQ ID NO: 71) | |
| | 9 | TGGAGTATGTGTCTGTGGAGACGA | 0.00004 |
| | | (SEQ ID NO: 72) | |
| | 9 | TGGAGTATGTGTCTGTGGAGACGAGA | 0.00004 |
| | | (SEQ ID NO: 73) | |

| | |
|---|---|
| forward primer | gcatttggttttaaattatggagtatg |
| | (SEQ ID NO: 5) |
| reverse primer | tgaaaaggccagttattccaa |
| | (SEQ ID NO: 14) |

TABLE 13
Analysis of EGFR T790M mutation

| cells and primers used | | | |
|---|---|---|---|
| tumor cell line | | | detection |
| used | | H1975 | sensitivity |
| blocking | 9 | AGCTCATCACGCAGCTCAT | 0.004 |
| primer | | (SEQ ID NO: 74) | |
| (overlapping | 18 | CCACCGTGCAGCTCATCAC | 0.004 |
| region, bp) | | (SEQ ID NO: 75) | |
| | 7 | CTCATCACGCAGCTCATGC | 0.02 |
| | | (SEQ ID NO: 76) | |
| | 6 | TCATCACGCAGCTCATGC | 0.02 |
| | | (SEQ ID NO: 77) | |
| | 11 | GCAGCTCATCACGCAGCTC | 0.0000001 |
| | | (SEQ ID NO: 78) | |
| | 8 | GCTCATCACGCAGCTCATGC | 0.0000002 |
| | | (SEQ ID NO: 79) | |
| | 13 | GTGCAGCTCATCACGCAGCTCAT | 0.0000001 |
| | | (SEQ ID NO: 80) | |

| | |
|---|---|
| forward primer | CACCGTGCAGCTCATCA |
| | (SEQ ID NO: 1) |
| reverse primer | cacatatccccatggcaaac |
| | (SEQ ID NO: 2) |

TABLE 14
Analysis of BRAF V600E mutation

| cells and primers used | | | |
|---|---|---|---|
| tumor cell line | | | detection |
| used | | SNU790 | sensitivity |
| blocking | 3 | AGCTACAGTGAAATCTCGATGG | 0.04 |
| primer | | (SEQ ID NO: 81) | |
| (overlapping | 9 | TGGTCTAGCTACAGTGAAATCTCG | 0.01 |
| region, bp) | | (SEQ ID NO: 82) | |
| | 17 | GGTGATTTTGGTCTAGCTACAGTGA | 0.001 |
| | | (SEQ ID NO: 83) | |
| | 11 | TTTGGTCTAGCTACAGTGAAATCTCG | 0.01 |
| | | (SEQ ID NO: 84) | |
| | 12 | TTTTGGTCTAGCTACAGTGAAATCTCG | 0.002 |
| | | (SEQ ID NO: 85) | |
| | 8 | GGTCTAGCTACAGTGAAATCTCGATGG | 0.0004 |
| | | (SEQ ID NO: 86) | |
| | 9 | TGGTCTAGCTACAGTGAAATCTCGATGG | 0.0004 |
| | | (SEQ ID NO: 87) | |
| | 10 | TTGGTCTAGCTACAGTGAAATCTCGATGG | 0.002 |
| | | (SEQ ID NO: 88) | |
| | 12 | TTTTGGTCTAGCTACAGTGAAATCTCGATGG | 0.002 |
| | | (SEQ ID NO: 89) | |
| | 10 | TTGGTCTAGCTACAGTGAAATCTCG | 0.02 |
| | | (SEQ ID NO: 90) | |
| | 6 | TCTAGCTACAGTGAAATCTCGATGG | 0.02 |
| | | (SEQ ID NO: 91) | |
| | 7 | GTCTAGCTACAGTGAAATCTCGATGG | 0.02 |
| | | (SEQ ID NO: 92) | |

| | |
|---|---|
| forward primer | CACAGTAAAAATAGGTGATTTTGGTCTAGC |
| | (SEQ ID NO: 54) |
| reverse primer | ctgatttttgtgaatactgggaact |
| | (SEQ ID NO: 61) |

As shown in FIG. 10, when the length of the overlapping sequence between the blocking primer and the generic primer was 1 or 2 bp, sensitivity was not high enough. Thus, the length of the overlap needs to be 3 bp or more.

5-7: Application of MEMO for Quantification and HRM (High-Resolution Melting Analysis)

MEMO may potentially be applied to quantitative real-time PCR and/or HRM analysis.

The present inventors performed HRM analysis combined with real-time PCR using a DNA-intercalating fluorescence dye for the samples of EGFR T790M mutation-containing DNA diluted with normal DNA at a dilution factor ranging from 1.0×10⁰ to 1.0×10⁻⁴.

A PCR reaction was performed using an AccuPower HF PCR PreMix (Bioneer) containing a hot-start, high-fidelity polymerase, buffer, and reagents (final concentrations: KCl 300 mM, MgCl₂ 25 mM and dNTP 0.3 mM). The reaction mixture contained 200 ng of DNA, 10 pmol of each generic primer and 50 pmol of the blocking primer (the amount of blocking primer was experimentally optimized to 50 pmol; FIG. 7). The PCR was performed using a 9600 thermal cycler (Applied Biosystems).

The PCR was performed under the following conditions (hereafter, same cycling conditions were used for detecting other mutations).

[PCR Cycling Conditions]
94° C. for 5 min (1 cycle); and then
50 cycles, each consisting of 30 sec at 94° C., 30 sec at 59° C., and 60 sec at 70° C.; and then
72° C. for 7 min (1 cycle)

HRM analysis combined with real-time PCR was performed using Rotor-Gene Q (Qiagen) in the presence of BEBO dye (TATAA Biocenter). Serially diluted DNA samples that contain EGFR T790M mutations were amplified using the blocking primer T790M-B6.

The analysis results show that changes in the threshold value of cycle (Ct) was within an acceptable range (0.6). The Ct values of the dilutions containing large amounts of mutant DNAs was lower than those containing small amount of the same. Consequently, the standard curve showed a linear correlation (r²=0.991; FIG. 16). That is, when the concentration of mutant DNAs is high, the threshold Ct values can be reached even with small number of cycles (Ct). But when the concentration of mutant DNAs is low, more cycles are needed to pass the threshold. In short, quantitative analysis of Ct values demonstrates that the present invention is useful.

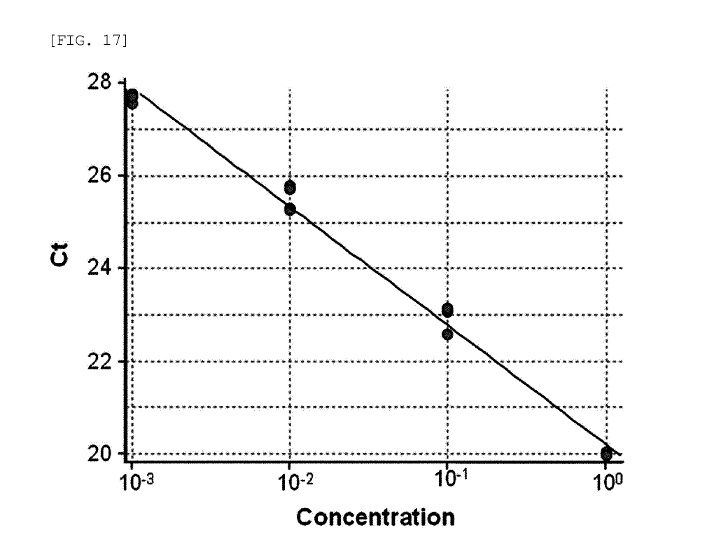

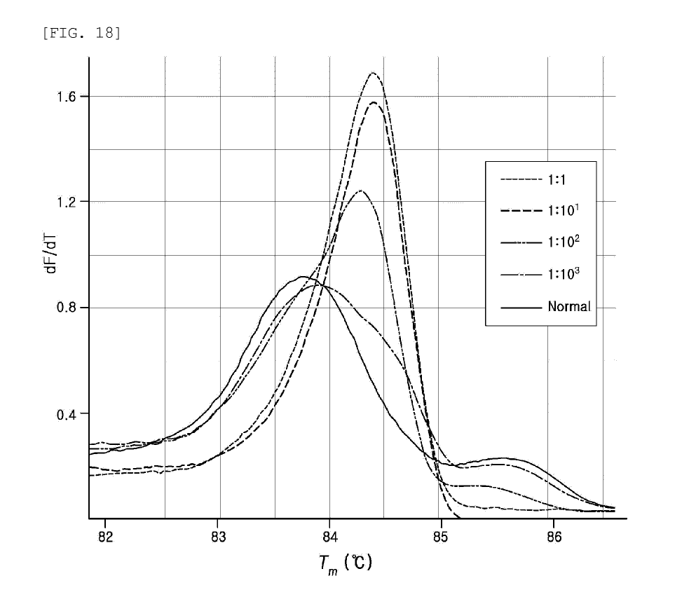

This linear correlation was evident at a dilution factor ranging from 1.0×10⁰ to 1.0×10³ (FIG. 17). This means that Ct value and concentration of mutant DNAs are in a linear correlation within this range. Therefore, the concentration of mutant DNA can be predicted by the corresponding Ct value. However, the linear correlation was not evident at dilution factor below 1.0×10⁻⁴. The efficiency of PCR was 1.45 which is lower than the general PCR efficiency, possibly due to the blocking of the amplification of the normal sequence.

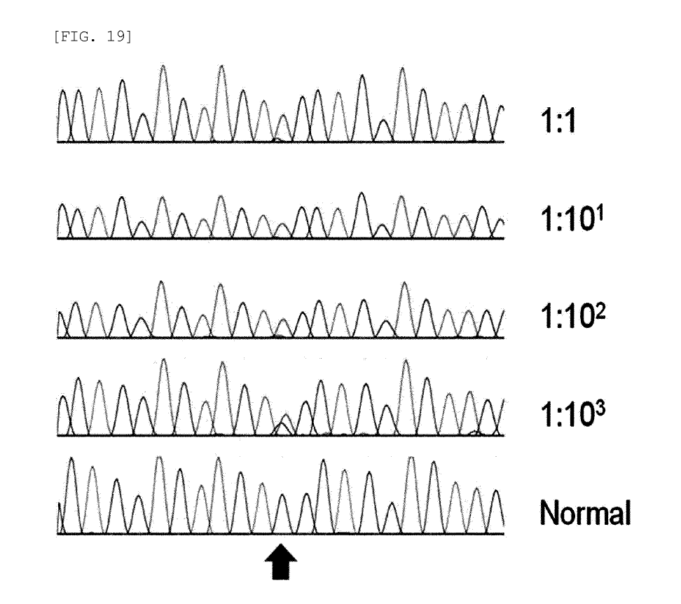

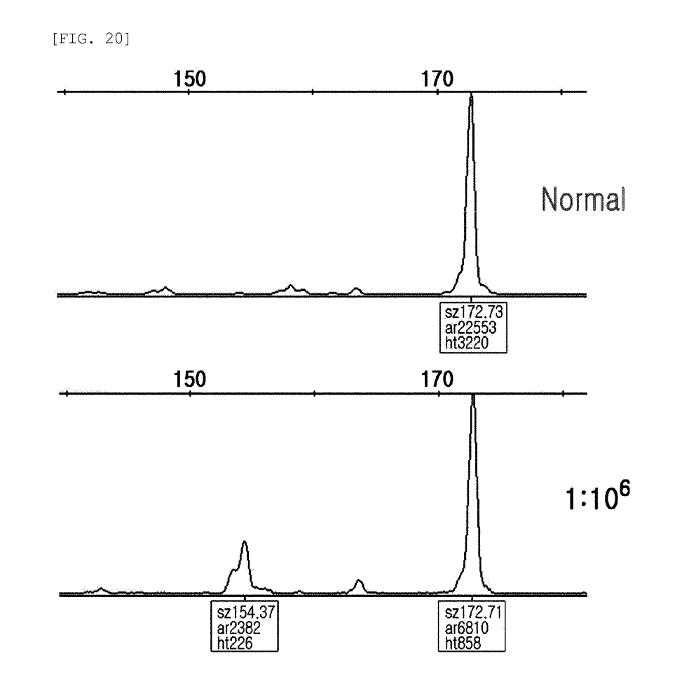

A small number of mutant alleles were amplified at the same or higher rate based on the comparison with normal alleles through MEMO-PCR. Thus the final products were suitable for HRM analysis. In the examples that used EGFR T790M mutation, dilutions containing larger amount of mutant DNA (1.0×10⁰, 1.0×10⁻¹ and 1.0×10⁻²) had a higher melting temperatures (Tm, 84.3-84.4° C.) than the normal samples (83.7° C.). This may be due to the greater stability of the mutant gene homoduplexes compared to normal gene duplex. Samples with a low concentration of mutant DNAs (<1.0×10⁻³) demonstrated heterozygous melting peaks complying with the sequencing results (FIGS. 18 and 19).

5-8: Improved Performance of Fluorescence PCR and Fragment Analysis

Small insertion and deletion mutations can be detected using size-based separation via fluorescence PCR and fragment analysis. The MEMO method was performed to detect two hot-focus mutations in EGFR and NPM1 genes.

Fluorescence PCR was performed for the 15-bp deletion in EGFR exon 19 and the 4-bp insertion in NPM1 exon 12. Generic primers (DEL15-F and NPM1-F) were 5′-FAM labeled. The amplicon fragments were analyzed according to size by the ABI Prism 3130xl Genetic Analyzer using GeneScan Software (Applied Biosystems).

The EGFR mutation is an important molecular markers for targeted treatment of lung cancers. And 50% of the mutation occurred in exon 19 is 15-bp deletion. The blocking primers were designed to encompass all of the known mutations in exon 19. And a highly sensitive primer sets were designed to detect a 1.0×10⁻⁶ dilution of minor alleles via downstream fluorescence fragment analysis. As a result, an abnormal peak that is 15-bp shorter than the normal peak appeared (FIG. 20).

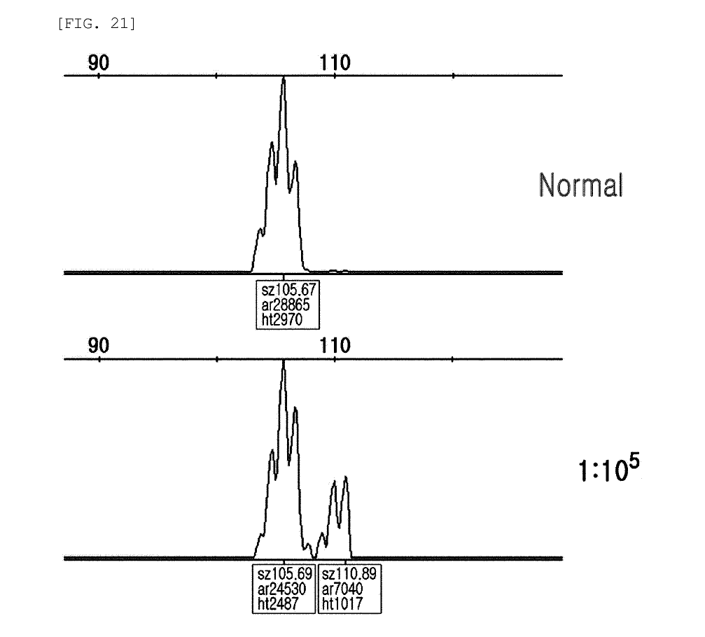

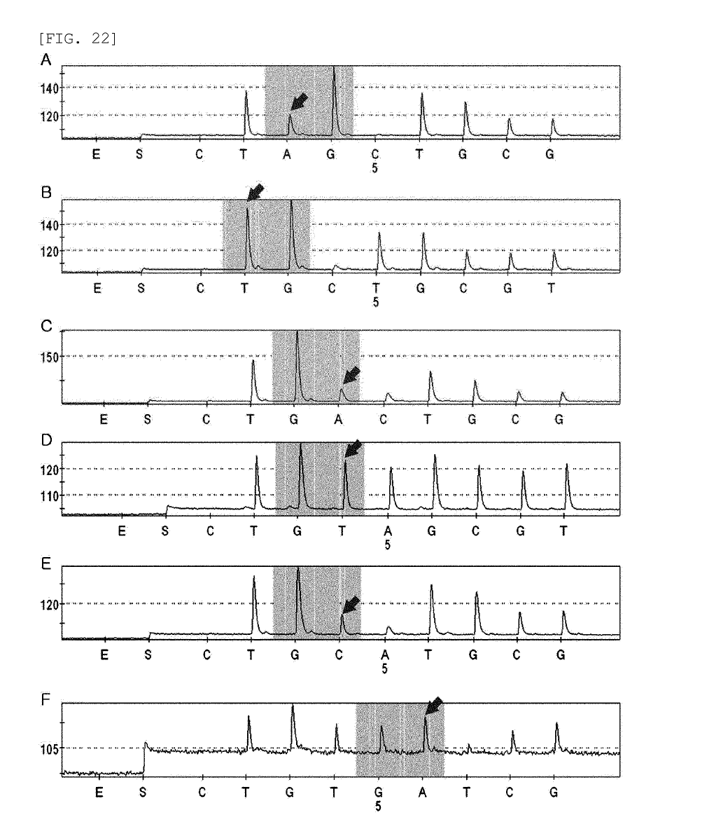

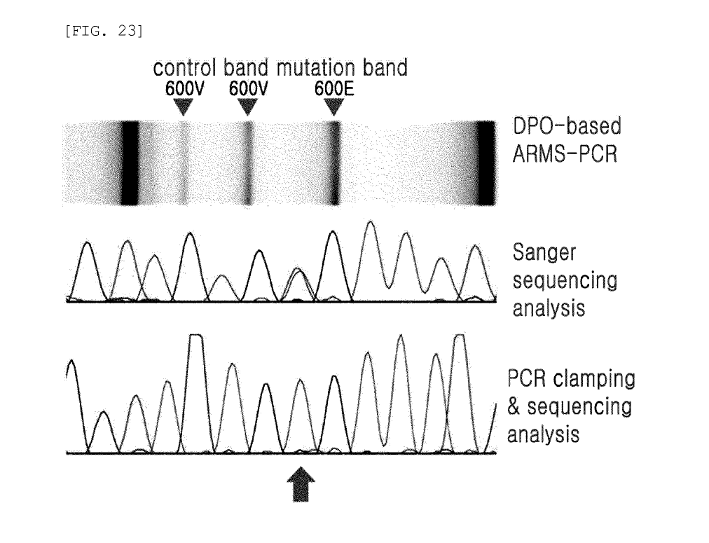

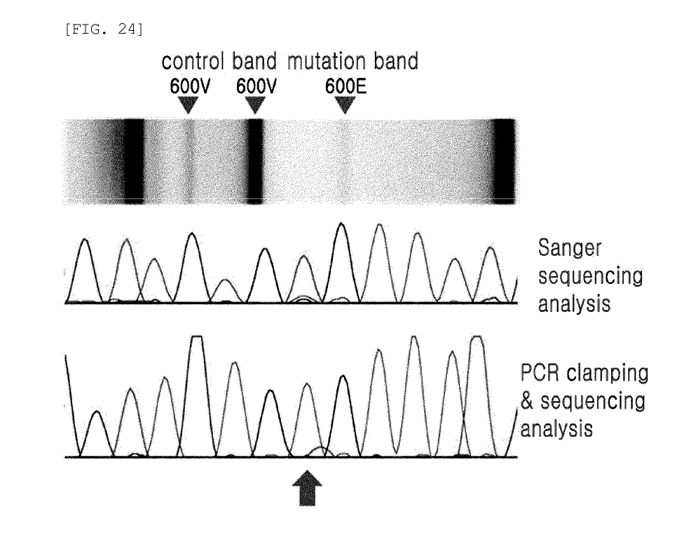

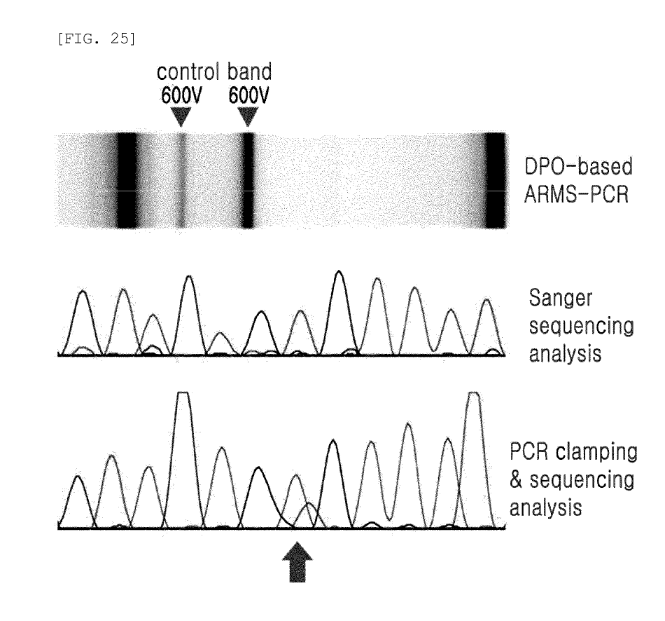

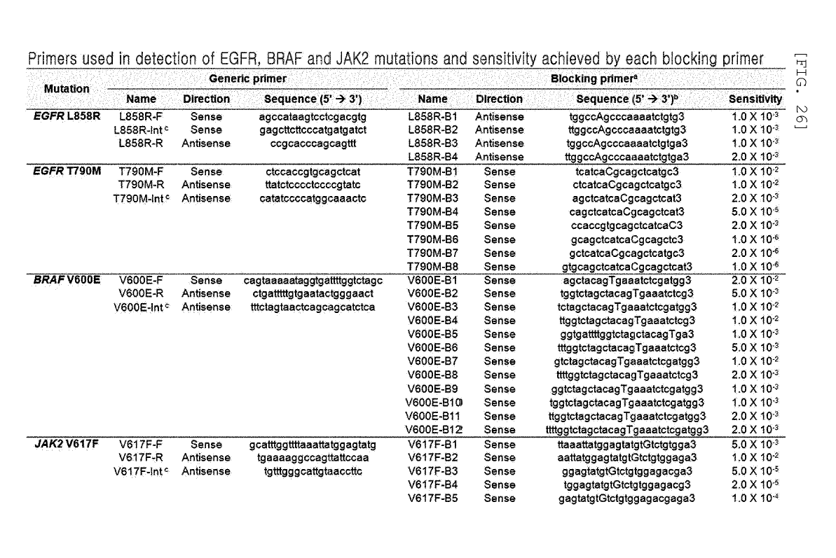

The NPM1 gene is the gene that is mutated most frequently in acute myeloid leukemia with a normal karyotype. These mutations typically results from an insertion of 4-bp in exon 12. The results of fluorescence PCR and fragment analysis showed an abnormal peak that is 4-bp longer than the normal peak (FIG. 21). It was confirmed that the best primer sets could detect mutations in concentrations up to 1.0×10⁻⁵ diluted sample of minority using MEMO-PCR and downstream fluorescence fragment analysis (FIG. 21).

5-9: Improved Pyrosequencing Performance

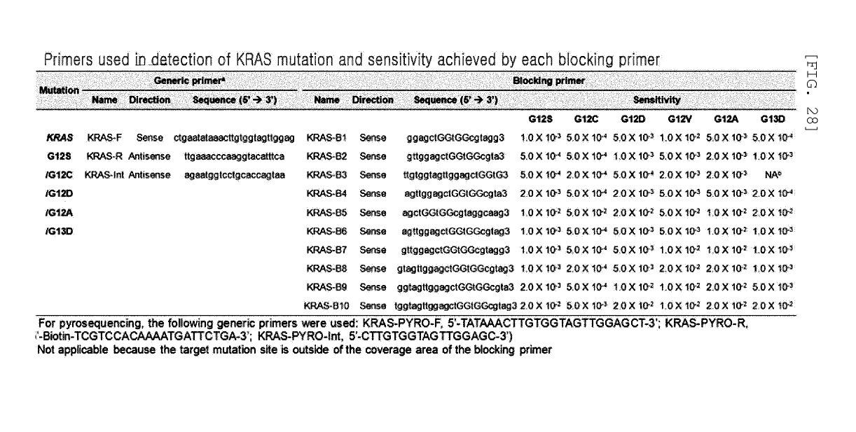

Pyrosequencing is a method for detecting sequence changes. In the present invention, it was used to analyze diluted samples having the KRAS mutation. The analysis was done through PSQ96MA (Biotage) instrument using the PyroMark Gold Q96 Reagents (Qiagen). The PCR reaction was performed using a blocking primer (KRAS-B2) and biotin-labeled generic primers (FIG. 27).

For diluted DNA samples containing KRAS G12S, G12C, G12D, G12V, G12A, and G13D mutations, the present inventors evaluated the MEMO method through pyrosequencing using a blocking primer and biotin-labeled generic primers. The observed sensitivities were 1.0×10⁻², 5.0×10⁻², 5.0×10⁻², 5.0×10⁻², 5.0×10⁻² and 2.0×10⁻² (FIG. 22), respectively, and no abnormal peaks were observed in the control sample with normal DNAs.

5-10: Clinical Verification and Comparison with Other Methods

To examine clinical applicability, DNA sample was extracted from 212 patients who were diagnosed to have thyroid nodules by ultrasonography. Cytological examinations were performed by specialized pathologists. DNA samples were analyzed by DPO-based ARMS-PCR and conventional PCR sequencing using a Seeplex BRAF ACE Detection kit (Seegene), as well as MEMO-PCR (using a V500E-B5 blocking primer) and downstream sequencing.

BRAF V600E mutations are observed in 50-90% of papillary thyroid carcinomas, and the molecular testing thereof is useful in diagnosis. Thyroid aspiration samples were collected from 212 patients who were found to have thyroid tumors in cytological examinations. Such samples were examined for BRAF V600E mutations by MEMO-PCR with downstream sequencing, DPO (dual-priming oligonucleotide)-based ARMS-PCR, and conventional PCR with downstream sequencing. The sensitivity of DPO-based ARMS-PCR for the detection of BRAF V600E was shown to be about 2.0×10⁻². MEMO-PCR including and sequencing analysis showed that all ARMS-PCR-positive samples were positive. It also detected mutations in 15 additional samples, which were not detected by ARMS-PCR and conventional PCR. Among the additional samples, 6 samples were found to be PTC, 4 samples were found to be indeterminate, and 5 samples were found to be nodular hyperplasia. Two of the four indeterminate samples underwent thyroidectomy and were found to be PTC by histology. One of the five nodular hyperplasia cases underwent thyroidectomy and was found to be follicular adenoma. One patient with PTC was found to be false-negative by ARMS-PCR but was positive in conventional PCR and MEMO-PCR with sequencing (Table 15). Therefore, it is evident that MEMO-PCR including sequencing has a higher sensitivity and specificity than ARMS-PCR and conventional PCR.

TABLE 15
Comparison between DPO-based ARMS-PCR, conventional PCR sequencing
and MEMO-PCR sequencing for detection of BRAF V600E mutations in
thyroid FNAC samples obtained from thyroid tumor patients.

| | Conventional | | Cytology | | | |
|---|---|---|---|---|---|---|
| DPO-based | PCR & | MEMO-PCR & | | | Benign/nodular | Total cases |
| ARMS-PCR | sequencing | sequencing | PTC | Indeterminate | hyperplasia | (n = 212) |
| + | + | + | 37 | | | 37 |
| + | − | + | 12 | | | 12 |
| + | − | − | | | 1 | 1 |
| − | + | + | 1 | | | 1 |
| − | − | + | 6 | 4^{b} | 5^{c} | 15 |
| NA^{a} | NA^{a} | + | | 1 | | 1 |
| − | − | − | 4 | 15 | 126 | 145 |

^{a}Not assessable due to test failure
^{b}Two cases underwent thyrectomy and were found to be PTC
^{c}One case underwent thyrectomy and was found to be follicular adenoma

As described above, the present invention provides a method for detecting mutant DNAs present in a small amount. This method can be used for diagnosing DNA mutation-related diseases such as tumors. Specifically, one embodiment of the present invention provides a technique for detecting mutant DNA with a high sensitivity and specificity by performing PCR using a blocking primer.

1. A composition for detecting mutant genes comprising a forward primer, a reverse primer and a blocking primer,
wherein the forward primer or the reverse primer that is closer to the mutation site comprises a nucleotide sequence complementary to the nucleotide sequence of the mutant gene that excludes the mutation site of the mutant genes in a sample;
wherein the blocking primer comprises a nucleotide sequence complementary to the wild-type sequence that corresponds to the mutation site of the mutant genes in the sample; one end of the blocking primer comprises the same nucleotide sequence as the inner end of the primer closer to the mutation site; and the other end of the blocking primer comprises a nucleotide sequence modified by the addition of one or more selected from the group consisting of C3-18 spacers, biotin, di-deoxynucleotide triphosphate, ethylene glycol, amine, and phosphate.

2. The composition of claim 1, wherein the composition is used to perform a polymerase chain reaction (PCR).

3. The composition of claim 1, wherein the distance between the mutation site and the primer closer to the mutation site is 1 to 9 base pairs (bp).

4. The composition of claim 1, wherein the molar concentration of the blocking primer is 1 to 50 times greater than that of the primer closer to the mutation site.

5. The composition of claim 1, wherein the melting temperature (Tm) of the primer closer to the mutation site is 55 to 65° C.; and the Tm of the blocking primer is 2 to 12° C. higher than that of the primer closer to the mutation site.

6. The composition of claim 2, wherein the annealing temperature of the PCR of the composition is lower than the melting temperature (Tm) of the wild type gene and blocking primer duplex, and is higher than the Tm of the mutant gene and blocking primer duplex.

7. The composition of claim 1, wherein the nucleotide sequence of the blocking primer that is the same as the inner end of the primer closer to the mutation site is 3 to 13 bp in length.

8. The composition of claim 1, wherein each of the forward primer and the reverse primer is consecutively 10 to 50 bp in length.

9. The composition of claim 1, wherein the blocking primer is consecutively 10 to 50 bp in length.

10. The composition of claim 1, wherein the mutation is a point mutation, an insertion of 1 to 50 bp, or a deletion of 1 to 50 bp.

11. The composition of claim 1, wherein the mutation is a tumor-specific mutation, a drug-resistance mutation in pathogenic bacteria or viruses, or a mitochondrial mutation.

12. The composition of claim 1, wherein the mutation is selected from a group consisting of EGFR T790M mutation, JAK2 V617F mutation, and KRAS G12D mutation.

13. A kit for detecting mutant genes comprising the composition of claim 1.

14. A method for detecting mutant genes comprising:
performing a polymerase chain reaction (PCR) on a gene sample containing the mutation site to be detected by using a forward primer, a reverse primer and a blocking primer; and identifying a mutation in the PCR product,
wherein the forward primer or the reverse primer that is closer to the mutation site comprises a nucleotide sequence complementary to the nucleotide sequence of the mutant gene that excludes the mutation site of the mutant genes in a sample;
wherein the blocking primer comprises a nucleotide sequence complementary to the wild-type sequence that corresponds to the mutation site of the mutant genes in the sample; one end of the blocking primer comprises the same nucleotide sequence as the inner end of the primer closer to the mutation site; and the other end of the blocking primer comprises a nucleotide sequence modified by the addition of one or more selected from the group consisting of C3-18 spacers, biotin, di-deoxynucleotide triphosphate, ethylene glycol, amine, and phosphate.

15. The method of claim 14, wherein the distance between the mutation site and the primer closer to the mutation site is 1 to 9 bp.

16. The method of claim 14, wherein the molar concentration of the blocking primer is 1 to 50 times greater than that of the primer closer to the mutation site.

17. (canceled)

18. (canceled)

19. The method of claim 14, wherein the nucleotide sequence of the blocking primer that is the same as the inner end of the primer closer to the mutation site is 3 to 13 bp in length.

20. The method of claim 14, wherein each of the forward primer and the reverse primer is consecutively 10 to 50 bp in length.

21. The method of claim 14, wherein the blocking primer is consecutively 10 to 50 bp in length.

22. (canceled)

23. (canceled)

24. (canceled)

25. A method for diagnosing mutation-related diseases using the method for detecting mutant genes according to claim 14.


SEQUENCE LISTING (93 sequences)

SEQ ID NO: 1 - DNA, 17 bp, Artificial Sequence
  forward primer for detecting EGFR T790M mutation
caccgtgcagctcatca

SEQ ID NO: 2 - DNA, 20 bp, Artificial Sequence
  reverse primer for detecting EGFR T790M mutation
cacatatccccatggcaaac

SEQ ID NO: 3 - DNA, 19 bp, Artificial Sequence
  blocking primer for detecting EGFR T790M mutation
gcagctcatcacgcagctc

SEQ ID NO: 4 - DNA, 28 bp, Artificial Sequence
  forward primer for detecting JAK2 V617F mutation
gcatttggttttaaattatggagtatgt

SEQ ID NO: 5 - DNA, 23 bp, Artificial Sequence
  forward primer for detecting JAK2 V617F mutation
caagcatttggttttaaattatg

SEQ ID NO: 6 - DNA, 26 bp, Artificial Sequence
  forward primer for detecting JAK2 V617F mutation
gcatttggttttaaattatggagtat

SEQ ID NO: 7 - DNA, 26 bp, Artificial Sequence
  forward primer for detecting JAK2 V617F mutation
agcatttggttttaaattatggagta

SEQ ID NO: 8 - DNA, 26 bp, Artificial Sequence
  forward primer for detecting JAK2 V617F mutation
aagcatttggttttaaattatggagt

SEQ ID NO: 9 - DNA, 25 bp, Artificial Sequence
  forward primer for detecting JAK2 V617F mutation
agcatttggttttaaattatggagt

SEQ ID NO: 10 - DNA, 25 bp, Artificial Sequence
  forward primer for detecting JAK2 V617F mutation
aagcatttggttttaaattatggag

SEQ ID NO: 11 - DNA, 25 bp, Artificial Sequence
  forward primer for detecting JAK2 V617F mutation
caagcatttggttttaaattatgga

SEQ ID NO: 12 - DNA, 25 bp, Artificial Sequence
  forward primer for detecting JAK2 V617F mutation
acaagcatttggttttaaattatgg

SEQ ID NO: 13 - DNA, 25 bp, Artificial Sequence
  forward primer for detecting JAK2 V617F mutation
cacaagcatttggttttaaattatg

SEQ ID NO: 14 - DNA, 21 bp, Artificial Sequence
  reverse primer for detecting JAK2 V617F mutation
tgaaaaggccagttattccaa

SEQ ID NO: 15 - DNA, 24 bp, Artificial Sequence
  blocking primer for detecting JAK2 V617F mutation
ggagtatgtgtctgtggagacgag

SEQ ID NO: 16 - DNA, 27 bp, Artificial Sequence
  forward primer for detecting KRAS G12D mutation
ctgaatataaacttgtggtagttggag

SEQ ID NO: 17 - DNA, 27 bp, Artificial Sequence
  forward primer for detecting KRAS G12D mutation
actgaatataaacttgtggtagttgga

SEQ ID NO: 18 - DNA, 27 bp, Artificial Sequence
  forward primer for detecting KRAS G12D mutation
gactgaatataaacttgtggtagttgg

SEQ ID NO: 19 - DNA, 29 bp, Artificial Sequence
  forward primer for detecting KRAS G12D mutation
aatgactgaatataaacttgtggtagttg

SEQ ID NO: 20 - DNA, 30 bp, Artificial Sequence
  forward primer for detecting KRAS G12D mutation
aaaatgactgaatataaacttgtggtagtt

SEQ ID NO: 21 - DNA, 22 bp, Artificial Sequence
  reverse primer for detecting KRAS G12D mutation
ttgaaacccaaggtacatttca

SEQ ID NO: 22 - DNA, 21 bp, Artificial Sequence
  blocking primer for detecting KRAS G12D mutation
tagttggagctggtggcgtag

SEQ ID NO: 23 - DNA, 17 bp, Artificial Sequence
  forward primer for detecting EGFR T790M mutation
caccgtgcagctcacac

SEQ ID NO: 24 - DNA, 17 bp, Artificial Sequence
  forward primer for detecting EGFR T790M mutation
caccgtgcagctcatca

SEQ ID NO: 25 - DNA, 17 bp, Artificial Sequence
  forward primer for detecting EGFR T790M mutation
ccaccgtgcagctcatc

SEQ ID NO: 26 - DNA, 17 bp, Artificial Sequence
  forward primer for detecting EGFR T790M mutation
tccaccgtgcagctcat

SEQ ID NO: 27 - DNA, 17 bp, Artificial Sequence
  forward primer for detecting EGFR T790M mutation
cctccaccgtgcagctc

SEQ ID NO: 28 - DNA, 20 bp, Artificial Sequence
  reverse primer for detecting EGFR T790M mutation
cacatatccccatggcaaac

SEQ ID NO: 29 - DNA, 19 bp, Artificial Sequence
  blocking primer for detecting EGFR T790M mutation
gcagctcatcacgcagctc

SEQ ID NO: 30 - DNA, 28 bp, Artificial Sequence
  forward primer for detecting JAK2 V617F mutation
agcatttggttttaaattatggagtatg

SEQ ID NO: 31 - DNA, 29 bp, Artificial Sequence
  forward primer for detecting JAK2 V617F mutation
aagcatttggttttaaattatggagtatg

SEQ ID NO: 32 - DNA, 31 bp, Artificial Sequence
  forward primer for detecting JAK2 V617F mutation
acaagcatttggttttaaattatggagtatg

SEQ ID NO: 33 - DNA, 32 bp, Artificial Sequence
  forward primer for detecting JAK2 V617F mutation
cacaagcatttggttttaaattatggagtatg

SEQ ID NO: 34 - DNA, 28 bp, Artificial Sequence
  forward primer for detecting KRAS G12D mutation
actgaatataaacttgtggtagttggag

SEQ ID NO: 35 - DNA, 28 bp, Artificial Sequence
  forward primer for detecting KRAS G12D mutation
gactgaatataaacttgtggtagttgga

SEQ ID NO: 36 - DNA, 29 bp, Artificial Sequence
  forward primer for detecting KRAS G12D mutation
gactgaatataaacttgtggtagttggag

SEQ ID NO: 37 - DNA, 29 bp, Artificial Sequence
  ATGACTGAATATAAACTTGTGGTAGTTGG
atgactgaatataaacttgtggtagttgg

SEQ ID NO: 38 - DNA, 30 bp, Artificial Sequence
  forward primer for detecting KRAS G12D mutation
aatgactgaatataaacttgtggtagttgg

SEQ ID NO: 39 - DNA, 29 bp, Artificial Sequence
  forward primer for detecting KRAS G12D mutation
tgactgaatataaacttgtggtagttgga

SEQ ID NO: 40 - DNA, 30 bp, Artificial Sequence
  forward primer for detecting KRAS G12D mutation
tgactgaatataaacttgtggtagttggag

SEQ ID NO: 41 - DNA, 31 bp, Artificial Sequence
  forward primer for detecting KRAS G12D mutation
atgactgaatataaacttgtggtagttggag

SEQ ID NO: 42 - DNA, 32 bp, Artificial Sequence
  forward primer for detecting KRAS G12D mutation
aatgactgaatataaacttgtggtagttggag

SEQ ID NO: 43 - DNA, 33 bp, Artificial Sequence
  forward primer for detecting KRAS G12D mutation
aaatgactgaatataaacttgtggtagttggag

SEQ ID NO: 44 - DNA, 34 bp, Artificial Sequence
  forward primer for detecting KRAS G12D mutation
aaaatgactgaatataaacttgtggtagttggag

SEQ ID NO: 45 - DNA, 16 bp, Artificial Sequence
  forward primer for detecting EGFR T790M mutation
ccaccgtgcagctcat

SEQ ID NO: 46 - DNA, 17 bp, Artificial Sequence
  forward primer for detecting EGFR T790M mutation
tccaccgtgcagctcat

SEQ ID NO: 47 - DNA, 17 bp, Artificial Sequence
  forward primer for detecting EGFR T790M mutation
ccaccgtgcagctcatc

SEQ ID NO: 48 - DNA, 17 bp, Artificial Sequence
  forward primer for detecting EGFR T790M mutation
cctccaccgtgcagctc

SEQ ID NO: 49 - DNA, 18 bp, Artificial Sequence
  forward primer for detecting EGFR T790M mutation
tccaccgtgcagctcatc

SEQ ID NO: 50 - DNA, 19 bp, Artificial Sequence
  forward primer for detecting EGFR T790M mutation
ctccaccgtgcagctcatc

SEQ ID NO: 51 - DNA, 19 bp, Artificial Sequence
  forward primer for detecting EGFR T790M mutation
cctccaccgtgcagctcat

SEQ ID NO: 52 - DNA, 27 bp, Artificial Sequence
  forward primer for detecting BRAF V600E mutation
agtaaaaataggtgattttggtctagc

SEQ ID NO: 53 - DNA, 28 bp, Artificial Sequence
  forward primer for detecting BRAF V600E mutation
cacagtaaaaataggtgattttggtcta

SEQ ID NO: 54 - DNA, 28 bp, Artificial Sequence
  forward primer for detecting BRAF V600E mutation
cagtaaaaataggtgattttggtctagc

SEQ ID NO: 55 - DNA, 29 bp, Artificial Sequence
  forward primer for detecting BRAF V600E mutation
tcacagtaaaaataggtgattttggtcta

SEQ ID NO: 56 - DNA, 30 bp, Artificial Sequence
  forward primer for detecting BRAF V600E mutation
ctcacagtaaaaataggtgattttggtcta

SEQ ID NO: 57 - DNA, 30 bp, Artificial Sequence
  forward primer for detecting BRAF V600E mutation
cacagtaaaaataggtgattttggtctagc

SEQ ID NO: 58 - DNA, 31 bp, Artificial Sequence
  forward primer for detecting BRAF V600E mutation
cctcacagtaaaaataggtgattttggtcta

SEQ ID NO: 59 - DNA, 31 bp, Artificial Sequence
  forward primer for detecting BRAF V600E mutation
tcacagtaaaaataggtgattttggtctagc

SEQ ID NO: 60 - DNA, 32 bp, Artificial Sequence
  forward primer for detecting BRAF V600E mutation
ctcacagtaaaaataggtgattttggtctagc

SEQ ID NO: 61 - DNA, 25 bp, Artificial Sequence
  primer for detecting BRAF V600E mutation
ctgatttttgtgaatactgggaact

SEQ ID NO: 62 - DNA, 27 bp, Artificial Sequence
  blocking primer for detecting JAK2 V617F mutation
ttaaattatggagtatgtgtctgtgga

SEQ ID NO: 63 - DNA, 24 bp, Artificial Sequence
  blocking primer for detecting JAK2 V617F mutation
tgtgtctgtggagacgagagtaag

SEQ ID NO: 64 - DNA, 27 bp, Artificial Sequence
  blocking primer for detecting JAK2 V617F mutation
tggagtatgtgtctgtggagacgagag

SEQ ID NO: 65 - DNA, 24 bp, Artificial Sequence
  blocking primer for detecting JAK2 V617F mutation
gagtatgtgtctgtggagacgaga

SEQ ID NO: 66 - DNA, 23 bp, Artificial Sequence
  blocking primer for detecting JAK2 V617F mutation
ggagtatgtgtctgtggagacga

SEQ ID NO: 67 - DNA, 26 bp, Artificial Sequence
  blocking primer for detecting JAK2 V617F mutation
ttatggagtatgtgtctgtggagacg

SEQ ID NO: 68 - DNA, 27 bp, Artificial Sequence
  blocking primer for detecting JAK2 V617F mutation
ttatggagtatgtgtctgtggagacga

SEQ ID NO: 69 - DNA, 23 bp, Artificial Sequence
  blocking primer for detecting JAK2 V617F mutation
tggagtatgtgtctgtggagacg

SEQ ID NO: 70 - DNA, 24 bp, Artificial Sequence
  blocking primer for detecting JAK2 V617F mutation
ggagtatgtgtctgtggagacgag

SEQ ID NO: 71 - DNA, 26 bp, Artificial Sequence
  blocking primer for detecting JAK2 V617F mutation
gagtatgtgtctgtggagacgagagt

SEQ ID NO: 72 - DNA, 24 bp, Artificial Sequence
  blocking primer for detecting JAK2 V617F mutation
tggagtatgtgtctgtggagacga

SEQ ID NO: 73 - DNA, 26 bp, Artificial Sequence
  blocking primer for detecting JAK2 V617F mutation
tggagtatgtgtctgtggagacgaga

SEQ ID NO: 74 - DNA, 19 bp, Artificial Sequence
  blocking primer for detecting EGFR T790M mutation
agctcatcacgcagctcat

SEQ ID NO: 75 - DNA, 19 bp, Artificial Sequence
  blocking primer for detecting EGFR T790M mutation
ccaccgtgcagctcatcac

SEQ ID NO: 76 - DNA, 19 bp, Artificial Sequence
  blocking primer for detecting EGFR T790M mutation
ctcatcacgcagctcatgc

SEQ ID NO: 77 - DNA, 18 bp, Artificial Sequence
  blocking primer for detecting EGFR T790M mutation
tcatcacgcagctcatgc

SEQ ID NO: 78 - DNA, 19 bp, Artificial Sequence
  blocking primer for detecting EGFR T790M mutation
gcagctcatcacgcagctc

SEQ ID NO: 79 - DNA, 20 bp, Artificial Sequence
  blocking primer for detecting EGFR T790M mutation
gctcatcacgcagctcatgc

SEQ ID NO: 80 - DNA, 23 bp, Artificial Sequence
  blocking primer for detecting EGFR T790M mutation
gtgcagctcatcacgcagctcat

SEQ ID NO: 81 - DNA, 22 bp, Artificial Sequence
  blocking primer for detecting BRAF V600E mutation
agctacagtgaaatctcgatgg

SEQ ID NO: 82 - DNA, 24 bp, Artificial Sequence
  blocking primer for detecting BRAF V600E mutation
tggtctagctacagtgaaatctcg

SEQ ID NO: 83 - DNA, 25 bp, Artificial Sequence
  blocking primer for detecting BRAF V600E mutation
ggtgattttggtctagctacagtga

SEQ ID NO: 84 - DNA, 26 bp, Artificial Sequence
  blocking primer for detecting BRAF V600E mutation
tttggtctagctacagtgaaatctcg

SEQ ID NO: 85 - DNA, 27 bp, Artificial Sequence
  blocking primer for detecting BRAF V600E mutation
ttttggtctagctacagtgaaatctcg

SEQ ID NO: 86 - DNA, 27 bp, Artificial Sequence
  blocking primer for detecting BRAF V600E mutation
ggtctagctacagtgaaatctcgatgg

SEQ ID NO: 87 - DNA, 28 bp, Artificial Sequence
  blocking primer for detecting BRAF V600E mutation
tggtctagctacagtgaaatctcgatgg

SEQ ID NO: 88 - DNA, 29 bp, Artificial Sequence
  blocking primer for detecting BRAF V600E mutation
ttggtctagctacagtgaaatctcgatgg

SEQ ID NO: 89 - DNA, 31 bp, Artificial Sequence
  blocking primer for detecting BRAF V600E mutation
ttttggtctagctacagtgaaatctcgatgg

SEQ ID NO: 90 - DNA, 25 bp, Artificial Sequence
  blocking primer for detecting BRAF V600E mutation
ttggtctagctacagtgaaatctcg

SEQ ID NO: 91 - DNA, 25 bp, Artificial Sequence
  blocking primer for detecting BRAF V600E mutation
tctagctacagtgaaatctcgatgg

SEQ ID NO: 92 - DNA, 26 bp, Artificial Sequence
  blocking primer for detecting BRAF V600E mutation
gtctagctacagtgaaatctcgatgg

SEQ ID NO: 93 - DNA, 25 bp, Artificial Sequence
  blocking primer for detecting BRAF V600E mutation
ggtgattttggtctagctacagtga